(19) United States
(12) Patent Application Publication
(10) Pub. No.: US 2011/0236874 A1
(43) Pub. Date: Sep. 29, 2011

(54) METHOD FOR FORMATION OF RENAL TUBULES

(76) Inventors: Daniele Zink, Singapore (SG); Jackie Y. Ying, Singapore (SG); Huishi Zhang, Singapore (SG); Farah Tasnim, Singapore (SG); Huishi Zhang, Singapore (SG); Farah Tasnim, Singapore (SG)

(21) Appl. No.: 13/132,594

(22) PCT Filed: Dec. 2, 2009
(86) PCT No.: PCT/SG2009/000463

§ 371 Date: Jun. 2, 2011

Related U.S. Application Data
(60) Provisional application No. 61/193,467, filed on Dec. 2, 2008.

(51) Int. Cl. A01N 1/00; C12Q 1/02; C12N 5/071

(52) U.S. Cl. 435/1.1; 435/29; 435/325

(57) ABSTRACT

There is provided a method of making a renal tubule. The method comprises seeding renal tubule cells onto a solid surface; culturing the renal tubule cells in a liquid growth medium to form a monolayer on the solid surface; and continuing culturing the renal tubule cells to form a tubule.

### CROSS-REFERENCE TO RELATED APPLICATION

This application claims benefit of, and priority from, U.S. provisional patent application No. 61/193,467, filed Dec. 2, 2008, the contents of which are incorporated herein by reference.

### FIELD OF THE INVENTION

The present invention relates generally to methods for forming renal tubules in gel-free in vitro systems.

### BACKGROUND OF THE INVENTION

Many drugs and xenobiotics (e.g. antibiotics, p-aminohippurate) cannot be cleared efficiently in the kidney by glomerular filtration. Such substances are then actively transported by the cells of the proximal tubule from the bloodstream into the glomerular filtrate flowing in the lumen of the renal tubule. Organic anion transporters, organic cation transporters and the p-glycoprotein coded by the multi drug resistance locus play a major role in such transcellular transport processes. Due to their functions in drug transport, proximal tubule cells are a major target for toxic drug effects in the kidney; as well, other cell types of the renal tubule can be affected by drugs in the filtrate. Thus, it is important to assess the nephrotoxic effects of newly developed drugs and their effects on the cells of the renal tubule.

There has been increasing interest in physiologic in vitro toxicity assays to replace animal experiments. The renal tubule is one of the major target sites in kidney injury caused by drugs, toxins and ischemia. Tubular necrosis is associated with acute and chronic kidney disease, and the renal tubule is affected and destroyed during kidney fibrosis, leading to end-stage renal disease. Thus, the renal tubule is one of the most interesting structures for examining drug effects and kidney disease.

Cell culture-based in vitro models employing tubular epithelial cells have been developed in order to study renal toxicity, drug effects, and kidney physiology and disease. Such models use mainly flat monolayer cultures of established animal or human cell lines, such as MDCK (Madin-Darby canine kidney), LLC-PK1 (Lewis-lung cancer porcine kidney 1), OK (opossum kidney) and its derivatives or HK-2 (human kidney-2).

The monolayer culture models have several disadvantages, due in part to the fact that the effect of a drug on such cells depends on cellular drug transport functions, which lead to cellular uptake. First, stable cell lines are functionally different from primary cells and the transport functions and responses to toxins may be altered. Second, animal cells do not always have similar transport properties and drug sensitivity as human cells. Third, transport properties and cell performance depends at least in part on the state of cell differentiation; and cells that are not fully or properly differentiated may not express certain drug transporters and may show altered drug responses. Proper cell differentiation is often not carefully controlled in studies using monolayer cultures, as such controls can be difficult to implement. Also, sub-confluent cell densities are often used; and under such conditions epithelial cells cannot form a differentiated epithelium, which expresses, for instance, tight junctions.

In the kidney, the cells of the renal tubule constitute a simple (single-layered) epithelium. The differentiated cells of the renal tubule epithelium display apical-basal polarity and the apical sides of proximal tubule cells (PTCs) possess a brush border with microvilli. Expression of brush border enzymes such as γ-glutamyl transpeptidase (γGTP) indicates proper cell type-specific differentiation and polarization of PTCs. Paracellular spaces within the renal tubule epithelium are sealed by tight junctions, which limit uncontrolled diffusion of substances between the compartments separated by the epithelium.

For investigating drug transport functions in vitro, renal tubule cells, including proximal tubule cells are typically grown on porous filters separating an apical and basal compartment of a cell culture well (e.g. Corning TRANSWELL™ plates). Most of these studies have been performed with the canine and porcine cell lines MDCK and LLC-PK1. For studying the functions of individual transporters, cells are transfected with corresponding cDNAs in order to overexpress the transporter proteins. The cells can then transport substances between compartments; if the concentrations in the different compartments change over time, cellular transport is presumed to be involved. Careful controls are required to ensure that 1) the cell layer is not leaky and that paracellular diffusion is not involved; and 2) the cells are well differentiated and perform those transport functions they would perform in vivo (if untransfected and primary cells are used).

Perfused kidneys (e.g. rat) or isolated renal tubules (human, mammalian animal, killifish) are also used as physiologic models in toxicity and uptake studies. The disadvantage of the perfused kidney/kidney tubule model is that larger numbers of animals must be sacrificed and that relatively laborious preparative work is required. Thus, the experiments are costly and laborious and not suitable for the testing and screening of larger numbers of compounds. Also, animal kidneys and renal tubules are physiologically different from human kidneys and renal tubules. The greatest disadvantage of isolated renal tubules is their limited lifetime, as they become functionally impaired after only a couple of hours.

In vitro, the formation of kidney tubules is studied using three dimensional (3D) gels. A 3D gel matrix is used as a support in which tubules can be grown in vitro (Karihaloo et al. Nephron Exp Nephrol 2005 100: e40-45; Lubarsky et al. Cell 2003 112: 19-28; Montesano et al. Cell 1991 67: 901-908; Montesano et al. Cell 1991 66: 697-711; Nickel et al. J Clin Invest 2002 109: 481-489; Sakurai et al. Proc Natl Acad Sci USA 1997 94: 6279-6284; Taub et al. Proc Natl Acad Sci USA 1990 87: 4002-4006; Zegers et al. Trends Cell Biol 2003 13: 169-176; Han et al. J Cell Sci 2004 17: 1821-1833). Some of these studies focused on MDCK cells, an immortalized canine cell line that is probably of distal tubule or collecting duct origin, grown in 3D gels consisting of collagen I and/or MATRIGEL™. Other of these studies involved primary rabbit renal proximal tubule cells embedded into 3D gels for studying tubulogenesis in vitro and the functional properties of the resulting tubules. Tubules formed in 3D gels (3D tubules) tend to be small and branched.

A general drawback of such 3D gel-based systems is that high-resolution imaging of intact functional tubules within gels is difficult. In addition, tubules embedded in 3D gels are difficult to access, and thus manipulations or applications of drugs cannot be performed in a well-controlled manner. These drawbacks limit the usefulness of in vitro 3D generated kidney tubules in functional studies and applications. One major area of interest for applications of in vitro generated kidney tubules are in vitro nephrotoxicity studies.

Renal tubules have also been formed on 2D solid surfaces (2D tubules) (Humes and Cieslinski Exp. Cell Res. 1992 201: 3-15; Takemura et al. Kidney Int. 2002 61:1968-1979; U.S. Pat. No. 5,429,938). Humes and Cieslinski have performed studies with primary rabbit renal proximal tubule cells and not with human cells. This reference addresses the role of growth factors and all-trans retinoic acid/laminin on tubule formation, but did not investigate functionality and detailed morphology of the 2D tubules. A corresponding US patent describes in vitro tubulogenesis occurring on 2D solid surfaces based on results obtained with rabbit renal proximal tubule cells. The obtained tubules are composed of thick aggregates of mainly non-polarized, adherent mesenchymal cells. Tiny, slit-like lumens form within these cell masses and the lumens have a width of less than one cell diameter. Only the few cells bordering these tiny lumens display epithelial differentiation and polarization. Thus, the tubule with its walls of epithelial cells is submerged within masses of mesenchymal cells.

It is not known whether the tubules described by Humes et al. are functional, which would be a prerequisite for any research on tubular functions or nephrotoxicology applications of such tubules, as the functionality has never been tested. The condensed cell masses surrounding the 2D tubules described by Humes et al. may interfere with studying tubular functions and with transport and toxicity assays. Large cell masses provide a problem with regard to high resolution imaging since the structures of interest are deeply buried within the cell aggregates. The penetration depth of conventional high resolution imaging techniques, such as confocal microscopy, is limited to about 15 μm and high resolution imaging of the interior of larger cell aggregates is problematic. As well, the tubule structures of interest are not directly accessible and, for example, nanoparticles applied to the medium cannot be delivered to the tubular epithelium in a controlled way.

Another report by Takemura et al. describes the formation of branched structures by immortalized genetically modified rat kidney epithelial cells on plastic surfaces. Although these structures were referred to as tubular-like, it was not investigated whether the tubules had a lumen.

### SUMMARY OF INVENTION

The present invention relates to a discovery that renal tubule cells can form renal tubules in vitro on solid surfaces. These tubules possess a lumen and may typically have a length in the range of from one to several millimetres (mm), even up to more than one centimetre. The cells forming the tubules tend to be well differentiated, with expression of epithelial and cell type-specific markers, and the tubules display functions specific for the tubular segment the cells were derived from.

The tubules provided by the present methods may be used as an in vitro model for studying tubular functions and transport processes occurring in tubular cells. Microscopic monitoring of the transport processes in the present tubules can be greatly facilitated due to the fact that the tubules may be easily imaged with high resolution using, for example, confocal microscopy, since the tubules are not formed within a gel matrix. As well, transcellular transport of fluorescent substrates may be more easily detected by fluorescent imaging of such tubules than by the use of current two-compartment systems that involve cell growth on porous membranes and measurement of substrate concentration in the different compartments. Transcellular transport can be easily detected by increases in the cellular and luminal levels of fluorescence and can be monitored online by using live cell microscopy.

The tubules may also be used as models for pathological processes affecting renal tubules, such as for example tubular necrosis. Tubular necrosis is frequently associated with kidney disease. The processes leading to tubular necrosis are not well understood, and can be difficult to address using in vivo models. The tubules of the present invention provide a convenient in vitro model not provided by monolayer cultures, and alterations in the tubule structure can more easily be examined than with tubules embedded in 3D gels.

The tubules provided by the present invention may also be used as in vitro models for toxicity assays, for example in the screening of drugs affecting tubular structure and functions and the subsequent analysis of drug-induced effects. Investigations of the interactions of nanoparticles with kidney tubules and of the effects of nanoparticles on kidney tubules may also be of interest, since nanoparticles often become enriched in the kidney. Nephrotoxic effects of nanoparticles have been observed but have not been studied systematically. Also, the kidney is involved in clearing nanoparticles from the body. However, nanoparticles above a certain size limit cannot be cleared by glomerular filtration. Whether other mechanisms as, for instance, tubular transport and secretion processes are involved in clearing larger nanoparticles form the kidney is currently unknown. Such studies may be of interest given the increasing number of applications of nanoparticles used in humans and the increasing presence of nanoparticles in the environment.

The tubules generated in vitro by the present methods using primary tubule cells, including, for example, primary human renal proximal tubule cells (HPTCs), may have several advantages compared to previously known in vitro model systems described above. Since they consist of primary cells, they are not at risk for functional alterations as with an immortalized cell line. If human primary tubule cells, including HPTCs, are used, then the tubules will have human physiology, which is not necessarily the case with non-human animal cells. As well, the tubules are surrounded by a closed and differentiated epithelium that does not tend to exhibit holes or gaps, as often observed in epithelia formed in monolayer cultures. Thus, transport experiments may require fewer controls for monolayer leakiness or proper cell differentiation, and the results may be easier to interpret. Additionally, the tubules generated by the present methods tend to be differentiated tissue-like structures surrounded by a differentiated epithelium displaying typical tubular transport functions. Thus, the differentiation status of the cells and associated functions are expected to be less variable than in experiments with monolayer cultures, which are not differentiated structures per se and in which cells can have various states of differentiation.

Thus, in one aspect, the invention relates to a method of making a renal tubule, the method comprising: seeding renal tubule cells onto a solid surface; culturing the renal tubule cells in a liquid growth medium to form a monolayer on the solid surface; and continuing culturing the renal tubule cells to form a tubule.

The renal tubule cells may be primary renal tubule cells, cells from a cultured cell line; embryonic primary kidney cells, kidney precursor cells, cells differentiated from embryonic stem cells, cells differentiated from mesenchymal stem cells or cells differentiated from induced pluripotent stem cells. The renal tubule cells may be proximal tubule cells, distal tubule cells or collecting duct cells. In particular, the primary renal tubule cells may be human primary renal proximal tubule cells.

The solid surface may be concave, may have an intersecting wall or may be a patterned surface having multiple intersecting walls. The solid surface may be coated with an extracellular matrix or an extracellular matrix component.

The liquid growth medium may comprise serum, including fetal bovine serum and for example, may comprise about 0.5% (v/v) or greater serum.

Optionally, a test compound may be added prior to tubule formation.

In another aspect, the invention relates to an in vitro renal tubule having one or more of the following properties: an unbranched or minimally branched morphology; a length of from about 0.1 mm to about 1.5 cm; an interior lumen surrounded mostly by differentiated epithelial cells; an interior lumen having a diameter of from about 1 μm to about 200 μm; and renal uptake and transport functions.

The in vitro renal tubule may be a human in vitro renal tubule. As well, the in vitro human proximal tubule may be prepared according to the method of the invention.

In another aspect, the invention relates to a method of monitoring tubular function, the method comprising: contacting a compound or particle with the exterior surface of an in vitro renal tubule prepared according to the method of any one of claims 1 to 10; and (i) detecting the compound or particle within the tubule; or (ii) assessing the effect of the compound or particle on the tubule or tubule cells; or both.

The compound or particle may be labeled with a detectable label.

The method may optionally include incubating the compound or particle with the tubule prior to detecting, and/or contacting an inhibitor with the exterior of the in vitro renal tubule.

The compound or particle may be a control compound or control particle, and the inhibitor may be a test inhibitor.

In yet another aspect, the invention relates to a method of assessing toxicity of a compound or a particle on a tubule, the method comprising: contacting a compound or particle with the exterior surface of an in vitro renal tubule prepared according to the method of the invention; and assessing the effect of the compound or particle on the tubule or tubule cells.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

The figures, which illustrate, by way of example only, embodiments of the present invention are described below.

FIG. 1. Micrographs showing histochemical detection of γGTP expression by HPTCs grown on collagen N, laminin, and collagen IV+laminin ECMs.

FIG. 2. Micrographs showing ZO-1 and α-SMA immunostaining patterns.

FIG. 3. Immunoblots and graphs depicting quantification of α-SMA expression by immunoblotting.

FIG. 4. Micrographs showing formation of cell aggregates.

FIG. 5. Micrographs showing different parts of a tubule of the present invention consisting of HPTCs.

FIG. 6. Micrographs showing different parts of a tubule of the present invention generated by LLC-PK1 cells

FIG. 7. Micrographs showing the morphology of tubules formed by HPTCs in MATRIGEL™.

FIG. 8. Micrographs showing the process of tubule formation on solid surfaces.

FIG. 9. Micrographs showing tubule formation over a time course.

FIG. 10. Micrographs showing tubules with a lumen lined by a differentiated epithelium.

FIG. 11. Micrographs showing organic anion transport.

FIG. 12. Micrographs showing tubule formation by HPTCs on solid surfaces and in 3D gels.

FIG. 13. Micrographs showing effect of solid surface architecture on tubulogenesis.

FIG. 14. Micrographs showing effect of solid surface architecture on tubulogenesis.

FIG. 15. Micrographs showing tubulogenesis in capillaries.

FIG. 16. Graphs and micrographs showing α-SMA expression in initial and 4 week-old cultures of HPTCs.

FIG. 17. Graphs and micrographs showing growth factor expression and effects of TGF-β1.

### DETAILED DESCRIPTION

Previously, in vitro kidney tubules have been generated employing three-dimensional (3D) gels, on the understanding that the formation of 3D tissue-like structures from in vitro cultivated cells requires a supporting 3D matrix. However, with the methods as described herein, the inventors have demonstrated that renal tubule cells, including primary human renal proximal tubule cells, can be cultivated in vitro on solid substrates to form large and functional kidney tubules, without the need for a supporting 3D gel matrix.

The mechanism used for tubulogenesis on 2D solid surfaces appears to be distinct from the mechanism employed in 3D gels; tubulogenesis on 2D solid surfaces involves interactions between epithelial and mesenchymal cells. The process involves transforming growth factor-β1, which is produced by kidney cells and is enhanced by a curved or walled substrate architecture. However, after triggering the process, the formation of renal tubules appears to occur with remarkable independence from the substrate architecture. The renal tubules generated on solid surfaces by the methods described herein typically have a length of several millimeters, and are easily accessible for manipulations and imaging. Thus, they are attractive for in vitro studies of renal tubule functions and nephrotoxicology.

Furthermore, the finding that cells organize into tissue-like structures independently from the substrate architecture may have important implications for kidney tissue engineering. Tubulogenesis on solid surfaces without a supporting 3D gel matrix may also allow for in vitro study of epithelial and mesenchymal cells interactions and regeneration of renal structures after organ disruption.

Thus, in one aspect there is provided a method for forming renal tubules. Renal tubule cells are seeded on a solid surface and grown to a monolayer in a liquid growth medium. The monolayer is grown on the solid surface under conditions that allow for cell growth and tubule formation.

The term “cell” as used herein refers to a single cell and is also intended to include reference to a plurality of cells, including a population, culture or suspension of cells, unless otherwise indicated. Similarly, the term “cells” refers to a plurality of cells, such as a population of cells, a cell culture or a suspension of cells, but is also intended to include reference to a single cell, unless otherwise indicated. A cell suspension refers to a liquid or semi-solid culture of cells, and a continuous cell suspension refers to a cell suspension of sufficient density to allow for cell-to-cell contact once the cells are deposited on a solid support.

The renal tubule cells may be any renal tubule cell type derived from a tubular structure or its precursor in the developing or fully developed kidney, including proximal tubule cells, distal tubule cells, or collecting duct cells. The renal tubule cells can be also derived from stem cells such as mesenchymal, embryonic or induced pluripotent stem cells, after application of appropriate protocols for differentiation in vitro. In a particular embodiment, the renal tubule cells are proximal tubule cells.

The renal tubule cells may be primary cells or may be cells from a cultured renal cell line (i.e. immortalized tubule cells). Primary renal tubule cells are cells directly explanted from an organism, including a mammal, including a human. Unlike renal tubule cells from a cultured cell line, primary cells have not undergone the process of immortalization or transformation, which process may alter characteristics of the immortalized tubule cells compared to primary renal tubule cells. In a particular embodiment, the renal tubule cells are primary renal tubule cells. In another particular embodiment, the renal tubule cells are cells from a cultured cell line, for example the porcine proximal tubule cell line LLC-PK1, the canine cell line MDCK or the opossum cell line OK and its derivatives.

The renal tubule cells, primary cells or from a cultured cell line, may be from any organism having a kidney, including a mammal, including a human.

In one particular embodiment, the renal tubule cells are primary human renal proximal renal tubule cells (HPTCs).

In the kidney, proximal tubule cells constitute a simple (single-layered) epithelium. The differentiated cells of the proximal tubule epithelium display apical-basal polarity and the apical sides possess a brush border with microvilli. Expression of brush border enzymes such as γ-glutamyl transpeptidase (γGTP) indicates proper cell type-specific differentiation and polarization of proximal tubule cells.

The renal tubule cells are seeded at a density sufficient to allow for formation of a monolayer on the solid surface. The renal tubule cells may be seeded at the density found within a monolayer or slightly below monolayer density.

For example, the renal tubule cells may be seeded at a density ranging from about 1×10³ cells/cm² to about 5×10⁵ cells/cm², or about 1×10³ cells/cm², about 1×10⁴ cells/cm², about 2×10⁴ cells/cm², about 3×10⁴ cells/cm², about 4×10⁴ cells/cm², about 5×10⁴ cells/cm², about 6×10⁴ cells/cm², about 7×10⁴ cells/cm², about 8×10⁴ cells/cm², about 9×10⁴ cells/cm², about 1×10⁵ cells/cm², about 2×10⁵ cells/cm², about 3×10⁵ cells/cm², about 4×10⁵ cells/cm², about 5×10⁵ cells/cm², about 2.65×10⁵ cells/cm². It will be appreciated that the lower the density below monolayer density, the longer it will take for the cells to form a monolayer and thus form the tubules. The cells should not be at such a low density so as to prevent monolayer formation.

Thus, the HPTCs are seeded onto the solid surface, including at a density slightly below monolayer density.

As used herein, a two dimensional surface (or 2D surface or 2D solid surface) refers to a surface that is not embedded within a gel matrix (or 3D gel). A two dimensional tubule (or 2D tubule) refers to a tubule grown or formed on a two dimensional surface. The 2D tubules are three dimensional in morphology, and the reference to 2D tubules is merely reference to tubules having the particular 3D morphology when grown on top of a solid surface. In contrast, a three dimensional tubule (or 3D tubule) refers to a tubule grown or formed within a gel matrix, and has a particular three dimensional morphology that arises from formation within a gel matrix.

In contrast to previously known methods that involve growth of tubules within gel matrices, the surface used in this method is a solid surface, meaning that the surface is sufficiently solid that the surface is not penetrated by cells or cellular outgrowths during tubule formation. The cells are seeded on top of the solid surface, form a monolayer on top of the solid surface and then organize into tubules on top of the solid surface. This is in contrast to a 3D gel matrix, which is semi-solid and which may be penetrated by cells or cellular outgrowth, or in which the cells may be directly embedded. 3D tubules may be formed either by mixing the cells into the gel before gellation, with the tubules then forming from cells embedded in the gel matrix, or by seeding the cells into pre-formed channels or passages formed in the gel matrix (typically in the range of about 100 μm (Schumacher et al. Kidney Int 2008; 73(10): 1187-1192.)), where the cells may form tubules or other structures. Typically, the diameter of tubules formed in 3D gels is in the range of 50 μm (see e.g. Han et al., J Cell Sci 117, 1821, 2004).

The solid surface may be an exposed or exterior surface, meaning it is not enclosed, for example the surface of a tissue culture plate or well or a glass slide or coverslip. The surface may be an enclosed or interior surface, for example the interior concave surface of a capillary tube, for example having a diameter of about 250 μm to about 750 μm, or about 550 μm to about 600 μm. It will be appreciated that use of an enclosed surface such as that within a capillary tube will result in a tubule that is less accessible, including for manipulation or imaging, than compared to a tubule formed on an exposed surface.

The solid surface may be any solid surface suitable to support cell growth. The surface may be flat, for example the surface of a glass slide or the bottom of a tissue culture well. The surface may have an intersecting wall meeting the surface, including at an obtuse angle, at an acute angle or at an orthogonal angle, for example at the edge of a tissue culture plate where the bottom of the plate meets the side wall. A patterned surface providing many intersecting wall structures, for example patterns of parallel channels or small wells with a flat bottom providing the solid surface may also be used to promote tubule formation. Alternatively, the surface may be concave, for example the interior surface of a capillary tube. Although the solid surface may be flat, use of a curved surface such as a concave surface or use of a flat surface with an intersecting wall may be more favourable for promoting tubule formation, meaning that tubules may form more quickly than on a flat surface with no intersecting wall.

The solid surface may be composed of any solid material that is capable of supporting cell growth, for example glass, borosilicate glass or a polymer such as used in tissue culture plates, including polystyrene and surface-treated polystyrene or polyester. For example, renal tubules may form on polyester membranes (PET, Transwell systems, Corning).

The dimensions of the solid surface influence the size of the resulting tubule in that it appears that the length of the tubule is constrained by the dimension of the surface along which the longitudinal axis of tubule aligns, with the tubule typically being shorter than the relevant dimension of the surface. Thus, if a circular coverslip is used as the solid surface, the length of the tubule formed will not equal the diameter of the coverslip, but rather the maximal length the tubule may reach will be shorter than the diameter of the coverslip.

The renal tubule cells are seeded onto the solid surface. In the present method, since the cells are seeded onto a solid surface, it is possible to culture the cells to form a confluent monolayer, which appears to be the first step in formation of the tubules by the present method.

The solid surface may optionally be coated with an extracellular matrix (ECM) or with an extracellular matrix component, prior to seeding of the HPTCs. As will be understood, extracellular matrix refers to an extracellular structure that anchors a cell layer, such as an epithelial layer, in vivo, which is secreted by certain cell types. In addition to the anchoring of cells, the ECM has important signaling functions and regulates cell behavior. In vivo, the extracellular matrix is made up of a complex mix of extracellular matrix proteins, including laminins, collagens including collagen I, III and IV, entactin, perlecan, proteoglycans, as well as heparan sulphate and other glycosaminoglycans and proteins. In vivo the composition of the ECM is specific for tissues and their substructures.

The extracellular matrix used may be an extracellular matrix secreted by a particular cell type, including HPTCs, or may be a commercially available matrix such as MATRIGEL™ Matrix, available from BD Biosciences, which is a matrix derived from the basal lamina produced by a murine tumour. MATRIGEL™ polymerizes at room temperature, and contains various basement membrane components and bound growth factors that are known to promote the establishment of epithelial tissues.

Alternatively, one or more components typically found in ECM and particularly in basal laminae may be used to coat the surface prior to depositing of the HPTCs. For example, one or more of laminin, collagen IV, collagen I, nidogen/entactin, perlecan, bamacan, agrin, tubulointerstitial nephritis antigen and nephronectin, or gelatine, which is derived from collagen. In particular embodiments, the solid surface is coated with a mixture of collagen IV and laminin, for example in a ratio of from about 10:1 to about 1:10 of collagen IV:laminin (w:w). In particular embodiments, from about 10.7 μg/ml collagen IV: 100 μg/ml laminin to about 150 μg/ml collagen IV: 100 μg/ml laminin may be used.

The type of ECM or ECM component may influence the time for tubule formation and may influence the rate and quality of the monolayer formed, as well as the extent of myofibroblast formation that occurs, which appear to be steps in the formation of the tubules, as described in more detail below.

To coat the surface with the ECM or ECM component, the ECM or ECM component may be diluted or dissolved in growth medium, added to the surface and then dried. For example, one or more ECM components may be solubilised in growth medium at a concentration for each of from about 5 μg/ml to about 1 mg/ml, prior to deposition on the surface and drying. If an extracellular matrix is used that is capable of forming a 3D gel, such as MATRIGEL™, the extracellular matrix should be applied as a thin surface coating and not as a 3D gel, in order to promote tubule formation on top of the solid surface and ECM coating.

Although the solid surface is such that the renal tubule cells do not penetrate the solid surface, the renal tubule cells may penetrate the ECM or ECM component coated on the solid surface. However, as noted above, the EMC coating should be applied in a thin layer so that formation of 3D structures by the cells within the coatings cannot occur and the cells always grow as a two dimensional monolayer, allowing tubule formation on top of the surface to occur (as opposed to embedded within a 3D gel matrix).

Once seeded onto the surface, the renal tubule cells are cultured in a suitable liquid growth medium. The liquid growth medium may be any growth medium that typically supports the growth of the renal tubule cells in culture, and contains required nutrients for growth, including salts, sugars and amino acids. For example, the growth medium may be a basal epithelial cell growth medium. The liquid growth medium is not gelled, solid or semi-solid, but is used in liquid form for the culturing of the seeded renal tubule cells.

The liquid growth medium may comprise serum as a component. As used herein, serum refers to the clear liquid portion of blood remaining after coagulation and removal of the cells and clotted protein. Serum includes any type of serum, including fetal bovine serum (FBS), newborn calf serum, donor bovine serum or human serum. In various embodiments, the growth medium comprises about 0.1% or greater serum, 0.5% or greater serum, about 1% or greater serum, or about 2% or greater serum, about 0.5% serum, about 1% serum, or about 2% serum. The percentages for serum concentration are given as % v/v. Generally, the lower the serum concentration included in the growth medium, the more slowly the renal tubule cells tend to grow. FBS was found to be important for tubule formation by HPTCs and in particular embodiments, the serum comprises or consists of fetal bovine serum and the growth medium comprises about 0.1% or greater FBS, 0.5% or greater FBS, about 1% or greater FBS, or about 2% or greater FBS, about 0.5% FBS, about 1% FBS, or about 2% FBS (all % v/v).

The liquid growth medium contains any other constituents or growth supplements required to support the survival and growth of the renal tubule cells. For example, the growth medium, which contains in addition about 0.5% to about 2.5% serum (for example FBS), may contain the following supplements: apo-transferrin (about 5-20 μg/ml), insulin (about 1-10 μg/ml), hydrocortisone (about 0.1-2 μg/ml), epinephrine (about 200-750 ng/ml), fibroblast growth factor 2 (FGF2) (about 1-3 ng/ml), EGF (about 2-20 ng/ml) and RA (about 0.1-100 nM), triiodothyronine (1-100 nM), L-Alanyl-L-glutamine (1-10 mM). For example, growth medium containing about 2% serum (e.g. FBS) may be supplemented with about 1% of epithelial cell growth supplement, which contains apo-transferrin (about 10 μg/ml), insulin (about 5 μg/ml), hydrocortisone (about 1 μg/ml), epinephrine (about 500 ng/ml), fibroblast growth factor 2 (FGF2) (about 2 ng/ml), EGF (about 10 ng/ml) and RA (about 10 nM).

Although transforming growth factor β1 (TGF-β1) is involved in tubule formation, supplementation with high concentrations of TGF-β1 at the time of seeding the cells on the solid surface may impair tubule formation or result in improperly formed tubules. Thus, the growth medium may be composed so it does not contain any specifically added TGF-β1. It will be appreciated that growth medium may contain very low or trace amounts of TGF-β1, as it may be difficult to fully purify other components from contaminating TGF-β1. For example, FBS contains trace amounts of TGF-β1. As well, kidney cells produce and secrete TGF-β1 themselves.

Alternatively, some TGF-β1 may be included in the growth medium, including addition at the time of seeding or after the monolayer has formed. For example, if TGF-β1 is added to the growth medium, it may be added to a concentration of from about 0.1 ng/ml to about 100 ng/ml.

The renal tubule cells may be cultured under suitable conditions to allow for monolayer formation and subsequent tubule formation. The cells are grown to a monolayer, which may be a confluent or closed monolayer, may exhibit tight junctions, and may be substantially free from holes or gaps. The monolayer may be well differentiated, meaning that most of the cells are differentiated to epithelial cells and display epithelial cellular markers, including for example ZO-1 and γGTP.

For example, the cells are cultured in the growth medium at an appropriate temperature (e.g. 37° C. for human cells), under an atmosphere of 5% CO₂, for between about 1 day to 4 weeks.

Following monolayer formation, the culturing of the cells is continued in the liquid growth medium until the cells re-organise and form tubules, as is discussed in detail below. Tubules may form after 1 day to 4 weeks in culture.

The timing and rate of tubule formation will be influenced by a number of different factors, including the cells used, the growth medium and supplements used, addition of TGF-β1, the architecture of the solid surface and any ECM or ECM component coated on the solid surface.

This method may be used to assess the effect of various compounds on the promotion or inhibition of tubule formation. A compound that is to be tested for effect on tubule formation may be added to the growth medium, including before seeding of the cells, before monolayer formation, following monolayer formation but before tubule formation, or during reorganisation of the cells for tubule formation.

The compound may be any compound of interest, for which it is desirable to determine if the compound has an effect on tubule formation, including promotion of tubule formation or inhibition of tubule formation. The compound may be a pharmaceutically active compound or a metabolite of a pharmaceutically active compound, for example a drug such as a small molecule compound.

In another aspect, there is provided an in vitro generated renal tubule (in the following referred to as in vitro renal tubule or 2D tubule). The in vitro renal tubules described herein are different in morphology than tubules formed in a 3D gel matrix. Thus, the present in vitro tubules, such as those formed on a solid surface, are also referred to as 2D tubules, to distinguish from tubules formed in a 3D gel matrix, referred to as 3D tubules. Thus, the in vitro renal tubule may be made on a solid surface, including using the above described methods.

The in vitro renal tubule may be any renal tubule that may be made from renal tubule cells, including a mammalian renal tubule, including a human renal tubule. As well, the in vitro renal tubule may be a proximal tubule, a distal tubule or a tubular structure formed by collecting duct cells. In a particular embodiment, the in vitro renal tubule is a human proximal renal tubule, and may be composed of primary human renal proximal tubule cells.

The tubule may have one or more of the following properties: a straight, unbranched or minimally branched (one or two branches per tubule) morphology; a length of from about 0.1 mm to about 1.5 cm; an interior lumen surrounded mostly by differentiated epithelial cells (that is, although some α-SMA expressing myofibroblasts may be present, the majority of cells lining the lumen will be differentiated epithelial cells); the lumen having a diameter of from about 1 μm to about 200 μm; the majority of the cells of the tubule being well differentiated, including epithelial cells; the epithelial cells exhibiting markers such as γGTP and ZO-1; renal uptake and transport function; associated with myofibroblast aggregates that express the α-SMA marker; may be continuous with an epithelial monolayer at one or both ends of the tubule.

The in vitro renal tubule tends to remain attached to myofibroblast aggregates, which may be associated with one or both ends of a tubule but may also be found at mid-tubular regions. Tubule ends that are not attached to a myofibroblast aggregate tend to be continuous with the remainder of the monolayer.

The in vitro renal tubules obtained by the above method display lumen formation, including lumens having a diameter in the range of about 1 μm to about 200 μm, about 10 μm to about 200 μm, or about 50 μm to about 200 μm. The tubule walls comprise differentiated epithelia expressing tight junctions and brush border markers. Some myofibroblasts are typically attached to these epithelia but do not tend to form condensed cell masses surrounding the tubules. Thus, the lining epithelia of the tubules are not submerged within other cell masses and are directly exposed to the environment.

The in vitro renal tubule exhibits functions similar to in vivo native renal tubules, including transporting organic anions into the lumen.

It has been observed that the above-described method results in generation of in vitro renal tubules that appear to form as follows. Upon seeding, the renal tubule cells form a flat and well differentiated epithelial monolayer. Some of the epithelial cells then appear to undergo epithelial-to-mesenchymal transition, resulting in increasing amounts of α-SMA-expressing myofibroblasts. The myofibroblasts form aggregates, and the epithelium surrounding the aggregates reorganises to form tubules. Thus, the tubules appear to result from a reorganised epithelial monolayer formed from renal tubule cells when seeded onto a solid surface.

Reorganisation appears to occur via highly coordinated and simultaneous directed movements of a large numbers of cells, resulting in retraction of the monolayer on one side of a myofibroblast aggregate and then the other side of the aggregate. These highly coordinated cell movements lead to the formation of stripes of cells, with the myofibroblast aggregates included within the stripes. Following stripe formation, the cells within the stripe then undergo additional rapid, dynamic reorganizations, resulting in tubule formation. Tubulogenesis on solid surfaces appears to be induced or at least influenced by TGF-β1, which is likely released in the in vitro system by myofibroblast aggregates. It is well documented that myofibroblasts release TGF-β1.

Thus, tubulogenesis on solid surfaces in the above-described methods appears to involve large-scale reorganizations of epithelial sheets around myofibroblast aggregates. Budding and branching morphogenesis, typically occurring in 3D gel matrices, does not appear to play a role and these processes are also not involved in renal tubule formation in vivo. In contrast, initial formation of differentiated monolayers does not occur in tubulogenesis that occurs in 3D gels and is thus not involved in tubule formation from the 3D gels. Individual cells typically first start to branch in 3D gels and outgrowth of cell cords occurs then from such branched cells or small groups of cells. The outgrowing cords, which have branched cells at their tips, then develop into tubules. Subsequent outgrowth of additional branches from the tubules tends to result in highly branched tubules. Branched cells are not observed during in vitro renal tubule formation on the solid surface and the in vitro renal tubules formed on solid surfaces are not highly branched, typically exhibiting either no branching or 1-2 branches per tubule.

In vivo, increased expression of TGF-β1 and appearance of myofibroblasts is a normal response to kidney injury. It is thought that myofibroblasts and TGF-β1 have important roles in tissue regeneration after injury apart from their prominent roles in fibrosis, which is difficult to study in vivo. Notably, functional tissue-like structures were re-organized in this in vitro system after the appearance of myofibroblasts and increased TGF-β1 expression. The finding that addition of TGF-β1 induced the initial steps of tubulogenesis on solid surfaces is in accordance with the results of a previous study performed with rabbit cells. However, the addition of TGF-β1 (beyond any trace contaminating amounts in the growth medium or that expressed by the myofibroblasts themselves) may affect tubule morphology, including the appearance of condensed masses of mesenchymal cells into which the tubule becomes submerged.

Thus, the human renal tubules generated on solid surfaces provided a useful in vitro model system. In contrast to tubules generated in 3D gels, the tubules generated on solid surfaces are easily accessible for manipulations, and administration of drugs, particles and other compounds of interest. The tubules formed on solid surfaces are exposed and thus can be readily imaged by high-resolution light microscopy and fluorescence microscopy. These properties make this in vitro model interesting for applications in tubular transport studies and in vitro nephrotoxicology. The described renal tubules might be a physiologically more relevant test system than monolayers of animal or human renal tubule cells, which are currently widely used for in vitro nephrotoxicology.

The accessibility of the in vitro renal tubules formed on solid surfaces for high resolution imaging allows detailed studies of the cellular and tubular transport processes by confocal and live cell microscopy. High-resolution imaging is not possible with tubules embedded into gels without sectioning, which destroys the tubules. Thus, live cell transport studies are very difficult with tubules formed in a 3D gel matrix. Imaging of transport processes and subcellular structures/processes would be important towards addressing renal tubule biology, function and pathology, and performing in vitro toxicity assays with drugs and nanoparticles.

Particles such as nanoparticles often become strongly enriched in the kidney after exposure in vivo, and particles above a certain size cannot be cleared by glomerular filtration. Whether and how such larger nanoparticles can be cleared from the kidney is not known. Thus, the tubules described herein may be useful for determining whether particles such as nanoparticles are transported into the renal tubules by the tubular cells and cleared in this way. This model system may be useful to systematically study uptake and tubular transport of nanoparticles, and the effect of the various features of the nanoparticles, such as size, shape, chemical composition, surface coatings, etc., on uptake and transport.

Thus, in another aspect, there is provided a method of monitoring tubule function, such as transport of a compound or particle. There is also provided a method of assessing toxicity of a compound or particle on a tubule.

The compound or particle is contacted with the exterior surface of an in vitro 2D renal tubule, such as a tubule formed by the methods described herein. The compound or particle of interest may be incubated with the tubule to allow for uptake, and transport by the tubule or to allow for the compound or particle to exert an effect on the tubule or cells, such as toxic effect. The tubule is then assessed to determine the location of the compound or particle or to assess the effect of the compound or particle the tubule or tubule cells, including the effect on tubular or cellular morphology and/or viability.

Monitoring tubular function includes detecting and/or assessing the uptake of a compound or particle within the cells of the tubule, the transport of a compound or particle that has been taken up by cells within the tubule to the lumen of the tubule, the effect of inhibitors on uptake and transport, and/or the effect including nephrotoxic effect and cytotoxic effect of a compound, particle or inhibitor on the tubular cells and the tubule, including on the morphology or survival of the tubule or tubular cells.

Thus, while the particle or compound may be monitored and detected to determine uptake and transport, the morphology of the tubule and the tubular cells, the degree of cell differentiation (epithelial) and trans-differentiation (myofibroblasts) of the tubular cells and the extent of cell death and cell viability may be also assessed; either with or without detecting the compound or particle that has been added. The compound or particle used may therefore be a compound or particle that is to be assessed for nephrotoxicity, or as a candidate for treatment of kidney disease, or for the ability to induce or prevent, inhibit or treat fibrosis (as indicated by altered numbers of alpha-SMA-expressing myofibroblasts), or to induce or inhibit tubule formation.

The compound or particle may be any compound or particle of interest, for which it is desirable to determine if the compound or particle is taken up by the cells of a tubule and transported by the tubular cells to the interior lumen of the tubule, or for which it is desirable to determine its nephrotoxic effects or its potential as drug for the treatment of kidney disease.

The compound may be a pharmaceutically active compound or a metabolite of a pharmaceutically active compound, for example a drug such as a small molecule compound. The compound may carry a charge. For example, the compound may be an anion, a cation, a zwitterion or may be uncharged. The compound may be any compound that is expected to be targeted for clearance by the kidneys, and thus may not be pharmaceutically active, for example such as a food additive, xenobiotics or other chemical that may be ingested by or internalised by a mammal, including a human.

The particle may be any particle that is expected to be inhaled or ingested by, implanted or injected into, or otherwise internalised in or by a mammal, including a human. For example, the particle may be a nanoparticle used in a medical treatment or that is the metabolic or degradation by-product of a substance used in medical treatment. The particle may also be a nanoparticle used in cosmetics, textiles or as food supplement or that is released in other ways into the environment.

The compound or particle may possess properties that allow for detection of the compound or particle directly. For example, the compound or particle may be coloured, fluorescent or radioactive. The particle may be of sufficient size to detect directly using known techniques such as dark field microscopy methods.

Alternatively, the compound or particle may include a detection label to assist with detection following potential uptake and possible transport by the tubule. The detection label may be any label that can readily be detected using known detection methods, for example a coloured label, fluorescent label, a radiolabel or a label that may be detected by an antibody or antibody fragment. Such labels are known and are readily available. For example, fluorescent labels include FITC, Rhodamine, TRITC, Texas Red, cyanine dyes (e.g. Cy3 or Cy5) or Alexa fluors.

The compound or particle is contacted with the exterior of the tubule. That is, the compound or particle is typically administered or delivered to the cells that form the outer layer of the tubule, and is not typically administered directly to the lumen of the tubule. For example, contacting may included adding the compound or particle to the growth medium surrounding the tubule.

Optionally, the particle or compound is incubated with the tubule for a desired length of time, for example from about 0.5 hours for about 24 hours, about 0.5 hours or more, or about 24 hours or less.

The contacting and optional incubating may be performed in the presence of an inhibitor of uptake or transport, to assess which particular pathway or pathways is or are involved in the uptake and/or transport of a compound or nanoparticle. The inhibitor or test inhibitor is contacted with the exterior of the tubule prior to, simultaneously with, or following the contacting of the compound or particle.

The inhibitor may be a known inhibitor or may be a compound that is to be tested for inhibition (i.e. a test inhibitor). If the inhibitor is a test inhibitor, the compound or particle referred to above may be a control compound or control particle, meaning a compound or particle that is known to be taken up by the cells of the tubule and possibly transported into the interior lumen of the tubule by a particular pathway. For example, the inhibitor or test inhibitor may be an inhibitor or potential inhibitor of an endocytotic pathway, or the p-aminohippurate transport system.

As with the compound or particle referred to above, the inhibitor or test inhibitor may be directly detectable or may be labelled with a detectable label. As will be appreciated, the inhibitor or test inhibitor should be distinguishable from the compound or particle.

Following contacting and optional incubating, the tubule may optionally be rinsed to remove excess compound or particle, or excess inhibitor or test inhibitor. This may be done by removing growth medium and replacing with fresh medium not containing the compound, particle, inhibitor or test inhibitor. The rinsing may be done one or more times, as desired.

The compound or particle is then detected within the tubule, using an appropriate detection method, such as fluorescence microscopy, epifluorescence microscopy, confocal microscopy, dark field microscopy, radiography, immunostaining or histochemistry.

Detecting the compound or particle within the tubule refers to detecting the compound or particle within the cells of the tubule following uptake of the compound or particle, as well as detecting the compound or particle within the lumen of the tubule following transport of the compound or particle.

Detecting may also involve sectioning of the tubules before and/or after performing the detection method, in order to better detect particles or immunodetection signal within the lumen of the tubule, or in order to assess the morphology of the cells and/or tubule after exposure to the compound, particle, inhibitor or test inhibitor.

If the compound or particle is not taken up by the tubule epithelial cells, the compound should not be detectable within the cells or the lumen of the tubules. The compound or particle may be taken up by the cells but not transported into the lumen, in which case the compound or particle should be detectable within the epithelial cells of the tubule. The compound or particle may be taken up by the epithelial cells and then transported into the lumen of the tubule, in which case the compound or particle should be detectable within the lumen of the tubule.

If an inhibitor or test inhibitor is used, a comparison may be done between assays performed with and without inhibitor.

The method may further comprising assessing the effect of the compound or particle, or the inhibitor or test inhibitor on the cells of the tubule, by assessing the morphology of the tubule. Such assessing may involve examining the cells or tubule for changes in morphology and may include sectioning the tubule and examining the interior lumen for changes in cellular or tubular morphology, including disruption or destruction of the tubule and the arrangement of cells within the tubule.

Assessing may also include determining the extent, if any, of cell death within the tubule, for example using known cell death detection assays, which may include staining with a compound that is taken up by dead cells and not by live cells.

Assessing may also include comparing the degree of differentiation of epithelial cells and determining the extent of transdifferentiation into myofibroblasts in the presence and absence of compound, particle, inhibitor or test inhibitor. For example, immunostaining for the α-SMA cellular marker that is present on myofibroblasts but not epithelial cells allows for the assessment of the number of myofibroblasts present in a tubule under specific conditions. As well, techniques such as immunostaining, fluorescent staining, or histochemical detection may be used to detect changes in ZO-1 expression patterns and/or detect brush border markers and other epithelial cellular markers.

In this way, any compound or particle, or inhibitor or test inhibitor, may be assessed for nephrotoxic effects. The uptake and transport of certain compounds, particles, inhibitors or test inhibitors may be toxic to the tubular cells, which toxicity may be manifested in changes or destruction of the tubule, as has been observed in vivo.

The described methods and tubules are further exemplified by way of the following non-limiting examples.

### EXAMPLES

### Example 1

ECM coatings were tested and the performance of primary human renal proximal tubule cells was monitored during extended periods of several weeks. During this process, it was observed that the formation of differentiated monolayers by human primary renal proximal tubule cells (HPTCs) was closely coupled to tubulogenesis, which occurred on solid surfaces. It was found that tubule formation is faster on ECMs consisting of collagen IV+laminin. The longer the monolayer is maintained, the later tubule formation occurs. This example relates to the experiments leading to tubule formation on solid surfaces.

Materials and Methods

Cell culture assay: All cell culture media used were also supplemented with 1% penicillin/streptomycin solution (ScienCell Research Laboratories, Carlsbad, Calif., USA), and all cells were cultivated at 37° C. in a 5% CO₂ atmosphere. HPTCs were obtained from ScienCell Research Laboratories and were cultivated in basal epithelial cell medium supplemented with 2% fetal bovine serum (FBS) and 1% epithelial cell growth supplement (ScienCell Research Laboratories). Cells were propagated in poly-L-lysine coated cell culture flasks. The seeding density at day 0 of the experiments was 5×10⁴ cells/cm². All experiments were performed with 24-well cell culture plates (Nunc, Naperville, Ill., USA) and cell culture medium was exchanged every two days in the experimental series.

Bone morphogenetic protein (BMP)-7 (Sigma Chemical Co, St. Louis, Mo., USA) was added at a concentration of 100 ng/ml. L-ascorbic acid 2-phosphate (AscP) (Sigma) was supplemented at a concentration of 50 μM. In the corresponding experimental series, cells were constantly kept in BMP-7 or AscP supplemented medium. In contrast, cells were treated with Trichostatin A (TSA, 10 ng/ml) (Merck, Darmstadt, Germany) overnight prior to seeding.

Live/dead assay: 2 μl of a 4′,6′-diamidino-2′-phenylindole (DAPI, Merck) solution (5 mg/ml) and 2 μl of a propidium iodide (PI, Invitrogen, Singapore) solution (1 mg/ml) were added to each well containing 1 ml of medium. Cells were kept in the incubator for 1-2 h before imaging.

Fixation: Fixation was performed with 3.7% formaldehyde in phosphate buffered saline (PBS) for 10 min at room temperature, followed by extensive washing with PBS. Fixed samples were always kept wet.

ECM coating: The commercially available pre-coated plates applied in some experiments were obtained from Becton and Dickinson (BD, Franklin Lakes, N.J., USA; BioCoat™ 6-Well Multiwell Variety Packs). The ECM coating of 24-well plates was performed by diluting the ECM components to the final concentration with cell culture medium. 100 μl of the coating solution was added to each well, and the plates were dried overnight in a laminar flow hood. Murine collagen I (750 μg/ml; Merck, Darmstadt, Germany), collagen IV (150 μg/ml, Merck) and laminin (100 pg/ml, Sigma) were used. Collagen IV and laminin were purified from human placenta, and in most experiments, they were employed at the afore-mentioned concentrations when applied in combination. However, one experimental series was performed with 10.7 μg/ml of collagen IV and 100 μg/ml of laminin. The complex ECM consisted of collagen IV (150 μg/ml) and laminin (100 μg/ml), as well as human recombinant nidogen (7.8 μg/ml) and nephronectin (7.8 μg/ml) (R&D Systems, Minneapolis, Minn., USA). In addition, poly-D-lysine (100 μg/ml, BD), poly-L-lysine (10 μg/ml, Sigma), pronectin F (Sanyo Chemical Industries, Kyoto, Japan) and gelatin (1 mg/ml, porcine type A; Sigma) were used.
Coating with MATRIGEL™ (BD) was performed according to the instructions of the manufacturer. MATRIGEL™ solution was diluted 70-fold with medium.

The ECM deposited by HK-2 cells was prepared or solubilized according to the literature methods (Beacham et al. Curr Protoc Cell Biol 2006; Supplement 33 (Chapter 10: Unit 10.9): 10.9.1-10.9.21). For cross-linking, the HK-2 ECM was incubated for 10 minutes with 3.7% formaldehyde. Afterwards, the cross-linked ECM was extensively washed with PBS.

Cell counting: Cells were trypsinized (0.05% trypsin/0.5 mM EDTA in PBS), resuspended in PBS and counted with a Beckman Coulter Particle Counter (Model Z1S; Beckman Coulter Inc., Fullerton, Calif., USA). For each ECM coating and time point, triplicates obtained from three different wells were counted.

Immunostaining, histochemistry, and imaging: Immunostaining was performed as described in Sadoni et al. J Cell Biol 1999; 146(6): 1211-1226. The following primary antibodies were used: rabbit anti-ZO-1 (zonula occludens-1, Invitrogen, Carlsbad, Calif., USA) and mouse anti-α-smooth muscle actin (SMA) (Abcam, Cambridge, UK). Alexa Fluor 488-conjugated anti-rabbit (Invitrogen) and TRITC-conjugated anti-mouse (Invitrogen) secondary antibodies were applied. After immunostaining, cell nuclei were stained with DAPI and cells were mounted with vectashield (Vector Laboratories, Burlingame, Calif.) for microscopy. Histochemical detection of γGTP activity and controls were performed as described in Ryan et al. Kidney Int 1994; 45(1):48-57. Imaging was performed with a Zeiss AxioObserver Z1 microscope (Carl Zeiss, Jena, Germany) using the Zeiss AXIOVISION™ imaging software.

Immunoblotting: For immunoblotting, cells were lyzed in 100 μl of heated NuPage LDS sample buffer (Invitrogen). Samples were collected, heated at 95° C. and centrifuged at 10,000 g for 2 minutes to pellet cell debris. Protein concentrations of the supernatants were measured with a NANODROP™ spectrophotometer (Biofrontier, Singapore). Appropriate amounts of the supernatants were loaded onto a NuPage precast gel (4-12%, Invitrogen) with the size marker PAGE Ruler Plus (Fermentas, Hanover, Md., USA). Proteins were transferred to iBlot membranes after electrophoresis. The membranes were blocked in TBS buffer containing 0.05% Tween 20 and 1% BSA (Sigma) at room temperature for 1 hour, and were then incubated overnight at 4° C. with 0.2 μl/ml of both mouse anti-α-SMA antibody (Abeam) and rabbit anti-α-tubulin (Abeam) antibody.

After washing, the membranes were incubated with 0.2 μl/ml of both peroxidase-conjugated sheep anti-mouse antibody and donkey anti-rabbit antibody. The blots were developed using the ECL detection kit (GE healthcare, Chalfont St. Giles, Buckinghamshire, UK) to produce a chemiluminescence signal captured on X-ray film. The films were scanned and analyzed using Adobe Photoshop CS3.

Sectioning of tubules: In order to obtain sections of tubules, HPTCs were grown on transwell polyester membranes (Corning Inc., Corning, N.Y., USA). After tubule formation, cells were embedded into TISSUETEK™ mounting medium and sectioned with a cryostat.

Calculations, statistics, and arrangement of images: Calculations and statistics (unpaired t-test) were performed using Excel 2003 software. Figures were arranged using AdobePhotoshop CS3 and ImageJ.

Figure Legends

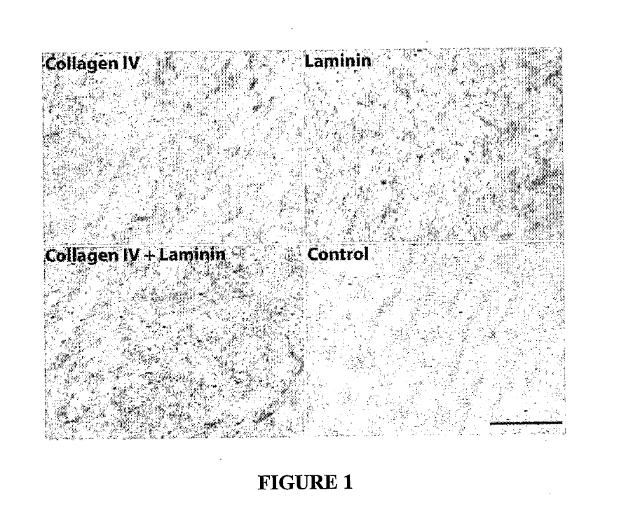

FIG. 1. γGTP expression. HPTCs were grown on collagen IV, laminin, and collagen IV+laminin ECMs as indicated, and γGTP activity was detected histochemically. γGTP activity results in red staining during incubation in reaction mixture. Control cells were grown on similar ECM coatings (shown for collagen IV), and incubated with reaction mixture lacking L-glutamic acid γ-(4-methoxy-β-naphthylamide). Scale bar 500 μm.

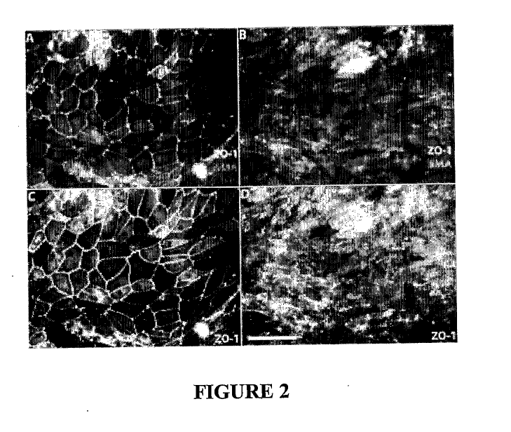

FIG. 2. ZO-1 and α-SMA immunostaining patterns. The upper panels show the merges of the different patterns (Z0-1: green, α-SMA: red, DAPI: blue) while the lower panels display only the ZO-1 immunostaining patterns. HPTCs were grown on an ECM consisting of (A, C) collagen N and (B, D) collagen N+laminin. Cells were (B, D) treated in addition with BMP-7 or (A, C) received no additional treatment. Scale bar 100 μm.

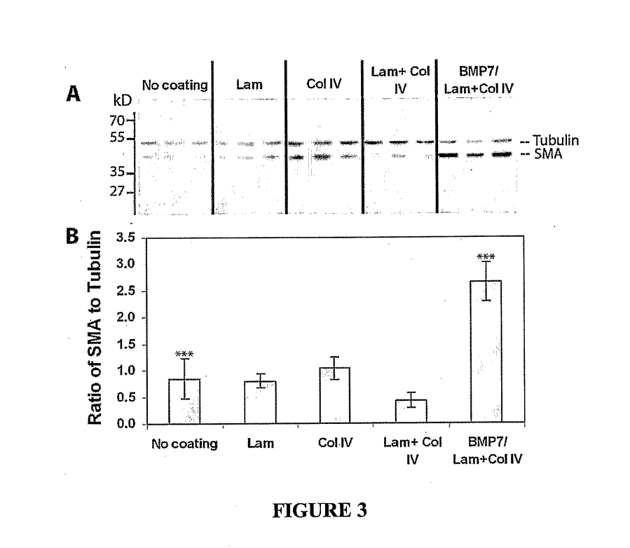

FIG. 3. Quantification of α-SMA expression by immunoblotting. (A) HPTCs were grown on uncoated TCP (control) or on the different coatings indicated (Col N: collagen IV, Lam: laminin). Cells grown on laminin+collagen N were either treated with BMP-7 or left untreated. Proteins were extracted in each case from 3 replicas, and the extract obtained from each sample was loaded onto a separate lane of the gel. α-SMA (lower bands, 42 kD) and α-tubulin (loading control, upper bands, 50 kD) were detected by immunoblotting. The positions of size marker bands and the corresponding molecular weights (kD) are indicated on the left. (B) The intensities of the bands shown in (A) were determined. The values obtained for the α-SMA-specific band of each lane were divided by the corresponding value obtained for the α-tubulin-specific band. The bars indicate the averages (±standard deviations) of these ratios for each coating and the control. Asterisks denote significant differences compared to the control, which are only observed after BMP-7 treatment (p=0.002).

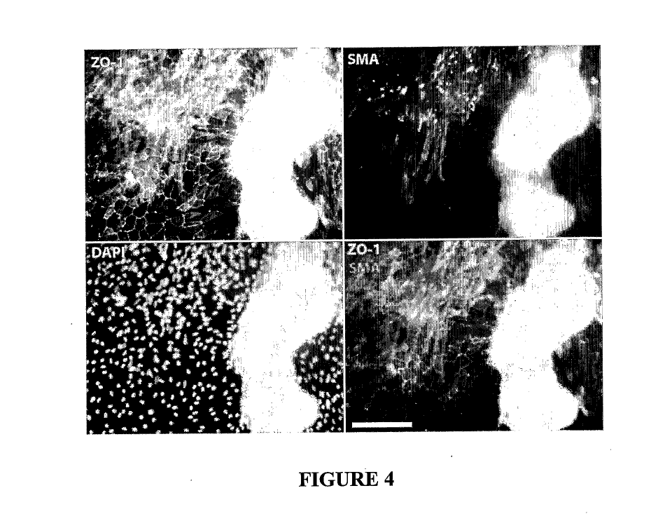

FIG. 4. Formation of cell aggregates. ZO-1 and α-SMA were detected by immunostaining, and the individual immunofluorescence patterns as well as the DAPI staining pattern (cell nuclei) are displayed. The merge is shown on the right (bottom; DAPI: blue, ZO-1: green, SMA: red). The upper left region of the imaged area was invaded by α-SMA-expressing myofibroblasts, which remained spread out on the substrate. In addition, α-SMA-positive cells formed a huge cell aggregate in the right half of the imaged area. The ZO-1-expressing epithelial sheet was folded up in areas where α-SMA-positive cells were present. Therefore, the ZO-1 staining patterns were out of focus in these areas. Scale bar 200 μm.

Results

Cell behavior was monitored over a period of four weeks in each experiment. During this period, assays for monitoring cell performance were performed weekly, starting one week after cell seeding. The initial seeding density was slightly below monolayer density. All experiments were conducted with multi-well plates wherein cells grew on ECM-coated tissue culture plastic (TCP).

The Performance of HPTCs in the Presence of Different ECMs and Additives

Experiments focused on monolayer and tight junction formation. A first test series (Table 1) was performed with non-coated (control) and with poly-L-lysine coated wells (poly-L-lysine is recommended for cultivation of HPTCs). This test series also included gelatin. As HPTCs might require a complex ECM more similar to the native ECM of the proximal tubule, and as such ECMs are difficult to assemble artificially, the effects of the ECM deposited by HK-2 cells was also tested.

TABLE 1
HPTC performance on different ECM coatings

| Coating/Treatment | Monolayer | ZO-1 |
|---|---|---|
| Non-coated | − | 0 |
| Poly-L-Lysine | − | 1 |
| Gelatin | +, until week 2 | 2-3 |
| Gelatin + AscP | +, until week 2 | 1-2 |
| Gelatin + BMP7 + AscP | + | 1-2 |
| HK-2 ECM deposited | + | 3 |
| HK-2 ECM solubilized | +, until week 3 | 3 |
| HK-2 ECM solubilized + AscP | +, until week 3 | 0-1 |
| HK-2 ECM solubilized + BMP7 + AscP | + | 1-2 |

In Table 1, formation (+) or no formation (−) of confluent monolayers is indicated. “until week x” means that confluent monolayers were formed after seeding, and remained intact at week x, but disintegrated afterwards. The rating of the ZO-1 staining patterns was performed independently from the assessment of the monolayers. The different types of ZO-1 immunostaining patterns (0-5) were classified as explained below.

Tight junction formation reduces the leakiness of the epithelium, which affects reabsorption, secretion and transport functions. The tight junctional protein ZO-1 is a well characterized marker expressed in differentiated epithelial cells. ZO-1 immunostaining patterns indicate the extent of tight junction formation. Proper formation of tight junctions between the lateral sides of the cells is indicated by a characteristic chicken wire-like ZO-1 immunostaining pattern.

As with the other assays performed, the state of epithelial differentiation and the extent of tight junction formation were monitored weekly by the immunostaining of ZO-1. Different types of ZO-1 staining patterns were observed. Diffuse cytoplasmic staining patterns not displaying any obvious lateral enrichments of ZO-1 were classified as type 0. Typical for the type I pattern were some dot-like lateral enrichments, while more extensive stretches of ZO-1 enrichments at some of the cell-cell interfaces were characteristic for a type 2 pattern. In these categories, all (0) or most (1 and 2) of the cell-cell contacts did not display any ZO-1 enrichments, indicating the absence of tight junction formation and chicken-wire like patterns.

Chicken wire-like patterns were characteristic for types 3-5. When chicken wire-like patterns remained restricted to some limited areas, the pattern was classified as type 3. In contrast, patterns were classified as types 4 or 5 when the entire cell layer displayed a chicken wire-like pattern. Some irregularity and minor disruptions were typical for a type 4 pattern. Very regular patterns were classified as type 5. Only types 4 and 5 immunostaining patterns indicated tight junction formation in extended areas, whereas types 0-3 patterns revealed that major areas of the epithelium were not sealed by tight junctions.

The results of the ZO-1 immunostaining experiments on HPTCs are summarized in Table 1. Cells were monitored over a period of four weeks, and immunostaining was performed weekly. Immunostaming was performed in parallel on two different samples for each time point and ECM coating. From each sample, several different randomly selected areas were imaged at a given time point, and for every time point and ECM, the most frequently occurring pattern was determined. From the patterns most frequently observed at a particular time point, Table 1 displays the best results obtained on a particular coating over the monitoring period of four weeks.

In control experiments, HPTCs were seeded directly onto the HK-2-deposited ECM after removal of the HK-2 cells. TCP coated with the HK-2 ECM after solubilization was also tested.

The ECMs and the combinations of ECMs and additives tested in the first experimental series with HPTCs are listed in Table 1. Cells remained separated from each other, and confluent monolayer formation was not observed on uncoated TCP and on a poly-L-lysine ECM.

In all other cases, confluent monolayer formation was observed, although disruption of the monolayer occurred in most cases during the second half of the monitoring period. The best results obtained with regard to tight junction formation were type 3 patterns, which were formed on directly deposited and on solubilized and coated HK-2 ECM in the absence of additives.

The results of further test series are listed in Table 2. The effects of the solubilized HK-2 ECM, were tested again but in the new test series, this ECM was fixed with formaldehyde after coating. This was done because ECM solubilization was achieved by treatment with guanidine-HCl, which is toxic and would require extensive washing after coating. Cross-linking with formaldehyde was performed in order to prevent ECM coating removal by washing. The new test series also included pronectin F and poly-L-lysine again as a negative control.

TABLE 2
HPTC performance and presence of α-SMA-positive cells

| Coating/Treatment | Monolayer | ZO-1 | α-SMA |
|---|---|---|---|
| HK-2 ECM, solubilized, fixed | +, until week 1 | 4 until week 2 | + |
| Poly-L-Lysine | −, but some | 5 at monolayer | + |
| | monolayer patches | patches and 2 at other | |
| | | areas | |
| Pronectin | Cells dead at week | Cells dead at week 1 | Cells dead |
| | 1 | | at week 1 |
| Collagen IV | +, until week 2 | 4-5 until week 3 | + |
| Laminin | +, until week 3 | 4-5 until week 3 | + |
| Laminin, 1% FBS and 0.25% | −, but some | 2 until week 3, 5 at | + |
| growth factor mix | monolayer patches | monolayer patches at | |
| | | week 4 | |
| Laminin + BMP7 | +, until week 2 | 3 until week 2 | + |
| Collagen IV + Laminin | +, but disrupted | 3 until week 4 | + |
| (10.7 μg/ml + 100 μg/ml) | before week 1 | | |
| Collagen IV + Laminin | +, until week 1 | 4-5 until week 1 | + |
| Collagen IV + Laminin + AscP | +, until week 3 | 2 until week 1 | + |
| Collagen IV + Laminin + BMP7 | +, until week 4 | 3 until week 1 | + |
| Collagen IV + Laminin + AscP + | +, until week 2 | 3 until week 1 | + |
| BMP7 | | | |
| Collagen IV + Laminin, 1% FBS | −, but some | 2 until week 3, 4 at | + |
| and 0.25% growth factor mix | monolayer patches | monolayer patches at | |
| | | week 4 | |
| Collagen IV + Laminin + | +, until week 2 | 4-5 until week 3 | + |
| Nidogen + Nephronectin | | | |

Disintegration of tight junctions after a certain time point is indicated. For example, “4 until week 2” means that a type 4 ZO-1 immunostaining pattern was present at week 1 and week 2, but the tight junctions disintegrated afterwards. Immunostaining with an antibody against α-SMA was performed in all cases. “+” indicates the presence of α-SMA-positive cells. For further explanations, refer to Table 1.

The new test series were mainly focused on collagen IV, laminin, and combinations of these components, since relatively good results were obtained with the corresponding ECMs with HK-2 cells (data not shown here, Zhang et al., 2009). The combinations of collagen IV and laminin were also tested in the presence of BMP-7 and AscP. In this test series, a laminin-rich combination of collagen IV+laminin (10.7 μg/ml and 100 μg/ml, respectively) was applied. Native basal laminae are laminin-rich and contain similar relative amounts of collagen IV and laminin.

Furthermore, in order to avoid possible overgrowth problems during the second half of the monitoring period, the laminin ECM and the combination of collagen IV and laminin was tested also in the presence of reduced amounts of growth factors. For this purpose, the concentrations of either FBS, or the epithelial cell growth supplement, or both components were gradually decreased. Various combinations were tested with HPTCs, and cell growth and morphology were assessed regularly by phase contrast microscopy. The best results were obtained with 1% FBS and 0.25% epithelial cell growth supplement (growth factor mix), and this combination was applied where indicated in Table 2. Furthermore, we included the complex ECM consisting of collagen IV, laminin, nidogen/entactin and nephronectin into the new test series.

The worst results were obtained with pronectin F, on which cells did not survive the first week. Some isolated patches of monolayers were obtained this time within poly-D-lysine coated wells at the end of the observation period, and extensive formation of tight junctions (type 5 pattern) occurred within these patches. However, on the majority of the poly-L-lysine coated surface area within a given well, cells remained isolated, did not form monolayers, and displayed type 2 ZO-1 staining patterns, in agreement with our previous results obtained with poly-L-lysine.

A confluent monolayer covering the entire bottom of the well was formed already during the first week on the HK-2 ECM fixed with formaldehyde after solubilization and coating. Extensive tight junction formation (type 4) was observed here. The best results in terms of tight junction formation were obtained with collagen IV, laminin, and with a combination of these components in the absence of additives. Similarly good results were also attained with the complex ECM consisting of four basal lamina components. In all of these cases, both type 4 and type 5 patterns were the most frequently observed patterns at all time points before disintegration occurred. Disintegration of tight junctions was observed after week 1 on the combination of collagen IV+laminin, and after week 3 on collagen IV, laminin and the complex ECM. Also the monolayers already disintegrated after week 1 on the combination of collagen+laminin, but were stably maintained until week 2 on collagen IV and the complex ECM, and until week 3 on laminin. Thus, in terms of monolayer and tight junction formation and maintenance, the best results were obtained with collagen IV, laminin, and the complex ECM.

Additional experiments showed that HPTCs grown on collagen IV and laminin ECMs as well as on collagen IV+laminin coatings expressed the brush border enzyme γGTP (FIG. 1), which provided further evidence that HPTCs were properly differentiated on these ECMs.

As the results showed, stable maintenance of a differentiated epithelial monolayer could be achieved for up to 3 weeks on collagen IV and laminin coatings. Formation of multiple layers and cell aggregates particularly during the second half of the monitoring period were observed. The monolayer detached at least from some areas, leading to the formation of areas devoid of cells.

Monolayer Disruption is Associated with the Presence of High Numbers of α-SMA-Positive Cells

Based on these observations, the question arose why the monolayer always disintegrated and could not be maintained for longer periods. It was also unclear whether all of the cells displayed proper epithelial differentiation, and whether cells at other states of differentiation were present. In particular the presence of myofibroblasts was of interest, which could arise by an epithelial-to-mesenchymal transition (EMT) process. Therefore, the presence of α-SMA-positive cells was monitored in the final experimental series. α-SMA is expressed in myofibroblasts but not in epithelial cells, and was detected in these experiments together with ZO-1 by co-immunostaining (FIG. 2).

Generally, α-SMA-positive cells were present in all samples tested (Table 2), and their numbers increased during the cultivation period. In the presence of low numbers of α-SMA-positive cells, the monolayer of epithelial cells connected by tight junctions was intact, and the α-SMA-expressing cells resided above or below the epithelial layer (FIG. 2A, C). However, in areas containing high numbers of α-SMA-expressing myofibroblasts, the epithelial monolayer was often disrupted, and the tight junctions were disintegrated (FIG. 2B, D). Typically, multiple layers of cells and cell aggregates were observed in such regions. Areas as shown in FIGS. 2A and B could be found simultaneously in the same well, and thus the effects were not globally occurring in a given well but were locally restricted, probably depending on the interaction of the different cell types.

Areas with high numbers of α-SMA-expressing cells as shown in FIG. 2B could be obtained with all ECMs and ECM/additive combinations tested. However, treatment with BMP-7 in particular led to the appearance of high numbers of α-SMA-expressing cells, associated with poor formation of tight junctions. The cell layer typically appeared irregular, and displayed multilayered areas frequently. Due to the multilayered nature of the areas containing many α-SMA-expressing cells, it was difficult to determine the cell numbers by using image analysis software. Also a quantitative analysis of the different cell types by flow sorting was difficult due to the presence of cell aggregates. Therefore, the extent of α-SMA expression was quantified by immunoblotting (FIG. 3). In agreement with the visual impression, a significantly (p 0.002) higher degree of α-SMA expression could be observed after BMP-7 treatment.

Monolayer Disruption is Associated with the Formation of Tubules on Flat Surfaces

Over the 4-week period, the occurrence of increasing numbers of α-SMA-positive cells coincided with the monolayers disruption. Disruption of the monolayers typically occurred during the second half of the monitoring period, and was associated with the disintegration of cell-cell contacts and tight junctions, the appearance of huge cell aggregates, and the detachment of the monolayer from the substrate, leading to areas devoid of cells. The question arose whether these processes and the appearance of α-SMA-expressing cells just reflected uncoordinated disintegration and damaging effects, which could be diminished by providing a more suitable in vitro environment, or whether such processes might be related to specific programs performed by the cells, which might have a different endpoint than monolayer formation.

To address this question, the areas containing multiple layers and the cell aggregates containing high numbers of α-SMA-positive cells were examined more closely. In the example shown in FIG. 4, the epithelial layer was still intact but folded up above the α-SMA-expressing cells. The epithelial cells were immobile and tightly connected to their neighbours as long as the typical epithelial junctions were present. In contrast, α-SMA-expressing myofibroblasts were mobile, and able to generate force with the contractile α-SMA-containing fibers. This suggested that the properties of the myofibroblasts would be required for the observed rearrangements of the epithelial layers, which was in agreement with the observation that epithelial layers were rearranged and folded up in areas where α-SMA-positive cells were present.

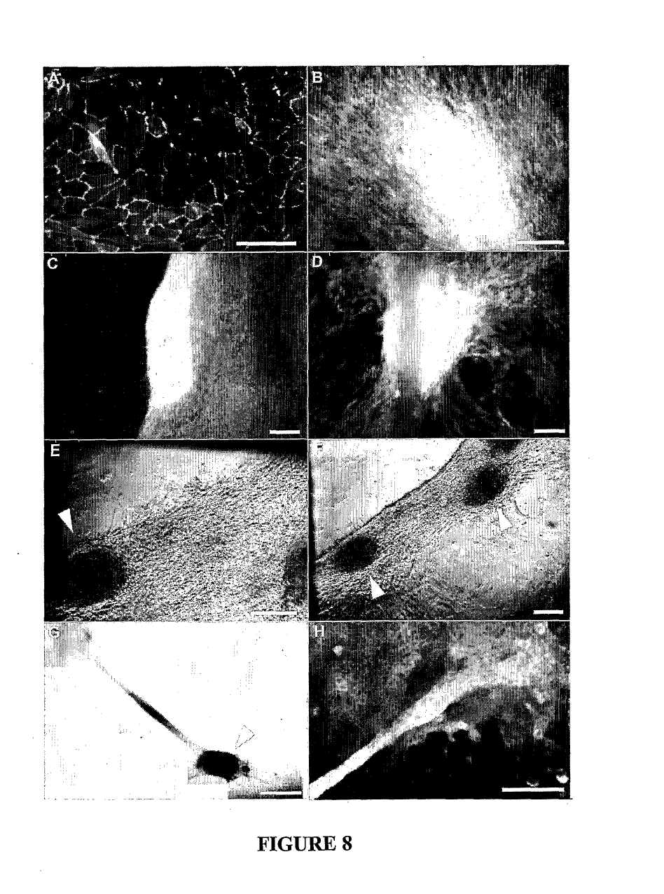

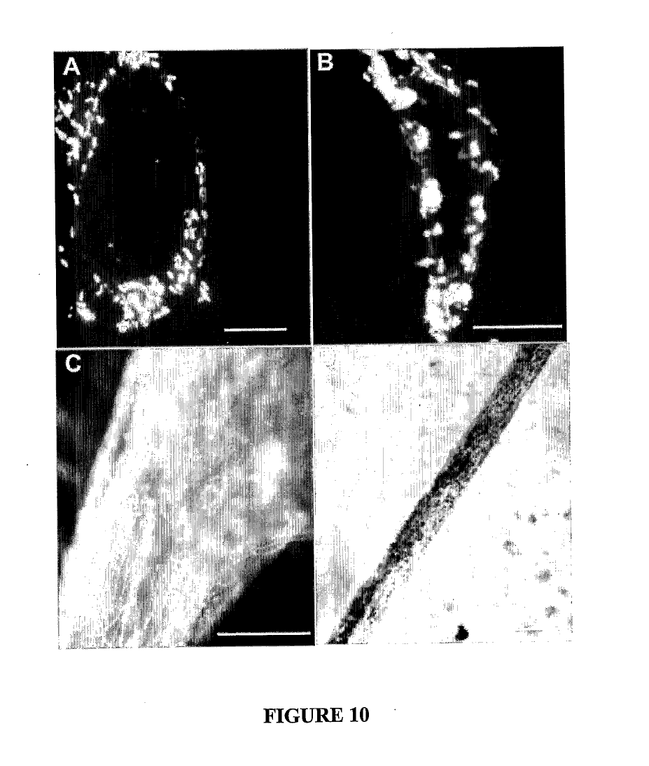

Although it was an intriguing possibility that α-SMA-positive cells played a crucial role in folding up and disrupting the monolayer, one could not exclude the possibility that this was just an uncontrolled and uncoordinated process due to the growth and accumulation of myofibroblasts below the epithelial sheet. Thus, the question arose whether this process was directed and led to a particular result. FIG. 8 H shows an area where the epithelial monolayer has been disrupted and folded up, resulting in a tubule-like structure sealed by tight junctions. Sectioning of such structures was performed to determine if they were cords or tubules with a lumen. Longitudinal sections (FIG. 10 B) confirmed that these structures consisted, at least partially, of a lumen lined by an epithelium expressing ZO-1. Together, the results showed that tubules consisting of differentiated epithelia were formed on flat two-dimensional (2D) solid surfaces, and this process was associated with monolayer disruption and detachment of cells from the substrate.

Discussion

Increasing numbers of α-SMA-positive cells were observed after the formation of differentiated monolayers, which probably arose by an EMT process. This was associated with disintegration and disruption of the monolayers and with the formation of tubules.

Since tubulogenesis of renal cells is usually studied by using cells embedded in three-dimensional (3D) gels it was surprising to find extensive tubule formation on 2D solid surfaces in the absence of a gel matrix.

### Example 2

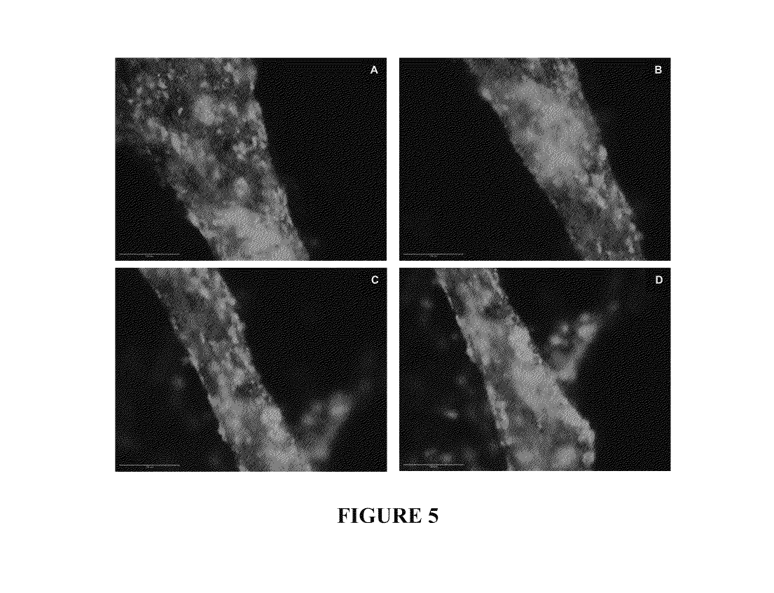

The four panels in FIG. 5 show different parts of a 2D tubule consisting of HPTCs (Scale bars: 100 μm).

First, histidine-coated quantum dots (QDs, green fluorescence) were added to culture medium. (DAPI counterstaining of cell nuclei: blue). 20 hours later the specimens were fixed and the 2D tubules were examined by fluorescence microscopy. The images show that the QDs were uptaken by HPTCs. There was no evidence for transport of the QDs into the tubular lumen (i.e. no enrichment of QDs in the lumen of the 2D tubule).

Next, transcellular transport of the QDs was examined using TRANSWELL™ plates where a monolayer of HPTCs grew on the lower side of a porous membrane separating two compartments (apical sides of cells facing the bottom compartment). QDs were added to the upper compartment. Occurrence of QDs in the bottom compartment of the TRANSWELL™ system (corresponding to the tubular lumen) could indicate transcellular transport. However, occurrence of QDs in the basal compartment could also be due to leakiness of the cell monolayer or accidental contaminations of the basal compartment with QDs during the experiments. The interpretation was somewhat complicated by the fact that the QDs were slightly toxic for HPTCs, which induced leakiness of the monolayer.

The results show that 2D tubules provide an excellent experimental system for addressing the question whether a certain substance becomes transported into the tubular lumen in order to clear it from the body.

The above experiment as described for the 2D tubules generated by HPTCs was repeated with 2D tubules generated from LLC-PK1 cells (porcine proximal tubule cell line frequently used for in vitro toxicology), using identical histidine-coated QDs.

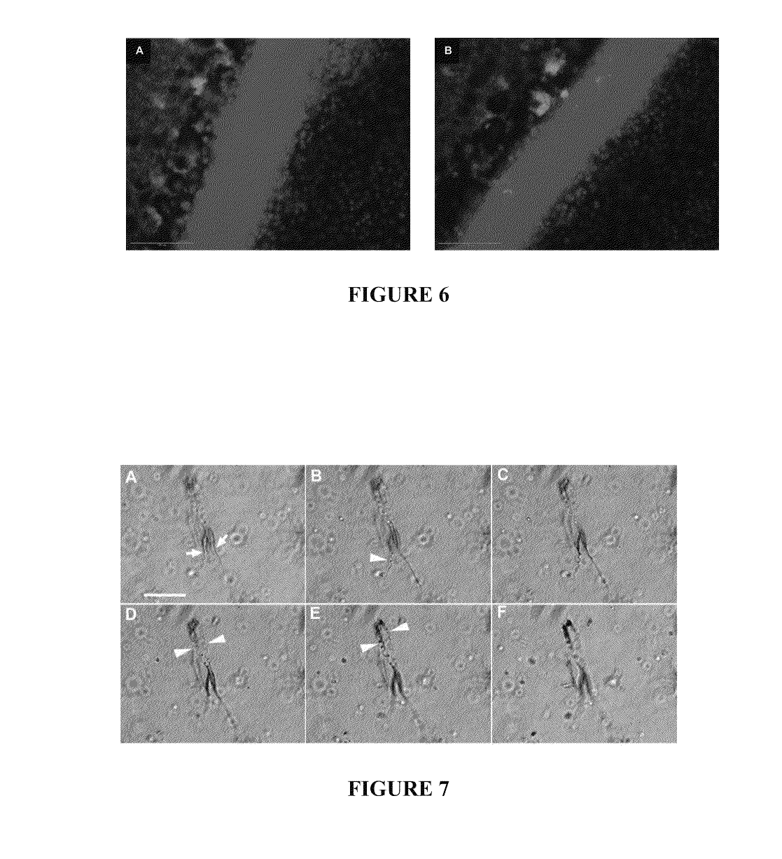

The two panels of FIG. 6 (Scale bars: 100 μm) show different parts of a 2D tubule generated from LLC-PK1 cells. The 2D tubule is the bright stripe in the middle of the image (blue: DAPI counterstain). A cell monolayer is on the right of the 2D tubule. The substrate surface on the left of the tubule is depleted of cells. The QDs (green fluorescence, same as used with HPTC 2D tubule) accumulated only in areas depleted of cells.

The results show that porcine LLC-PK1 cells and primary human proximal tubule cells interact differently with the same kind of QDs. This underlines the importance of using primary human cells and 2D tubules formed by HPTCs for in vitro nephrotoxicology and nanotoxicology.

### Example 3

### Materials and Methods

Cell culture: Different batches of HPTCs were obtained from ScienCell Research Laboratories (Carlsbad, Calif., USA). Cells were cultivated in basal epithelial cell medium supplemented with 2% fetal bovine serum (FBS) and 1% epithelial cell growth supplement (ScienCell Research Laboratories). In some experiments, TGF-β1 (R&D Systems, Minneapolis, Minn., USA) was added at a concentration of 10 ng/ml after monolayer formation.

Cells were cultivated on uncoated multi-well plates (Nunc, Naperville, Ill., USA) or plates coated with human laminin or other ECMs as described in Zhang et al. Biomaterials 2009 30: 2899-2911. The seeding density was 5×10⁴ cells/cm², unless otherwise indicated.

The wells of diagnostic printed slides have a diameter of 2 mm and a glass bottom. No ECM coating was applied in experiments using the printed slides.

Cells were seeded at a density of 2.65×10⁵ cells/cm² into glass capillaries (inner diameter=0.58 mm; Sutter Instrument, Novato, Calif., USA) and kept in static culture. Only the ends of the capillaries were examined.

Applications of MATRIGEL™ (BD, Franklin Lakes, N.J., USA) were performed according to the manufacturer's instructions.

Sectioning of tubules: HPTCs were grown on polyester TRANSWELL™ membranes (Corning. Lowell. MA, USA) (pore size=0.4 μm). The membranes with the tubules were embedded in TISSUETEK™ O.C.T. (Sakura Finetek, Tokyo, Japan) and sectioned.

Immunostaining and histochemistry: Histochemical detection of γ-glutamyl transpeptidase (γGTP) activity, formaldehyde fixation and immunostaining were performed as outlined in Zhang et al. Biomaterials 2009 30: 2899-2911.

Transport assays: Tubules formed 1-2 weeks after seeding were cultivated in phenol red-free medium supplemented with 80 μM of lucifer yellow (Sigma Aldrich Chemical Corp, Singapore), 10 μM of rhodamine 123 (Invitrogen, Singapore), 5 μM of 5,6-carboxydichlorofluorescein diacetate (5,6-carboxyfluorescein) (Invitrogen) or 5 μM of BODIPY FL verapmil (Invitrogen). Tubules were fixed after 20 h of incubation, and subsequently stained with 4′,6′-diamidino-2′-phenylindole (DAPI).

RNA Isolation and Reverse Transcription Procedures: Total RNA was isolated using TRIZOL™ reagent (Invitrogen). Three replicas were analyzed for each time point. The RNA was purified using the RNEASY™ Mini Kit (Qiagen, Hilden, Germany). The RNA SUPERSCRIPT™ III RTPCR kit (Invitrogen) was employed for reverse transcription.

qRT-PCR was performed by using the ICYCLER™ system and software (BioRad, Hercules, Calif., USA). Gene expression levels were calculated relative to the expression levels of the house keeping gene glyceraldehyde 3-phosphate dehydrogenase (GAPDH) using the BioRad software.

Primers used to determine expression levels of the indicated genes are set out in Table 3.

TABLE 3
Forward and Reverse Primers used
in qRT-PCR experiments

| | | SEQ ID |
|---|---|---|
| Probe | Sequence | NO. |
| GAPDH forward | ATCACCATCTTCCAGGAGCGA | 1 |
| GAPDH reverse | CCAAAGTTGTCATGGATGACC | 2 |
| α-SMA forward | TCATCACCAACTGGGACGAC | 3 |
| α-SMA reverse | ATGCTCTTCAGGGGCAACAC | 4 |
| TGF-β1 forward | ACCTGAACCCGTGTTGCTCT | 5 |
| TGF-β1 reverse | GCTGAGGTATCGCCAGGAAT | 6 |
| LIF forward | TACGCCACCCATGTCACAAC | 7 |
| LIF reverse | TGTCCAGGTTGTTGGGGAAC | 8 |
| FGF2 forward | TGTGCTAACCGTTACCTGGC | 9 |
| FGF2 reverse | GCCCAGGTCCTGTTTTGGAT | 10 |
| KGF forward | AGTGGCAGTTCGGATTGTGG | 11 |
| KGF reverse | CCCCTCCATTGTGTGTCCAT | 12 |
| HGF forward | TGTCCACGGAAGAGGAGATG | 13 |
| HGF reverse | AGCCCCAGCCATAAACACTG | 14 |

Imaging, statistics and software: Imaging was performed with a Zeiss AXIOOBSERVER™ Z1 microscope (Carl Zeiss, Jena, Germany) using the Zeiss AXIOVISION™ imaging software. Calculations and statistics (unpaired t-test) were performed using Excel 2003. Figures were arranged with AdobePhotoshop CS3 and ImageJ.

Figure Legends

FIG. 7: Morphology of 3D tubules formed by HPTCs in 3D gel matrix of MATRIGEL™. The panels show different focal planes of a branched tubular structure. The branched structure comprises convoluted tubules (marked by arrowheads) and straight tubules. Thinner tubules are continuous with wider lacunae in the middle of the structure. Intersections between tubules and lacunae are marked with arrows. Scale bar: 100

FIG. 8: The process of tubule formation on 2D solid surfaces. Panels A-D and H show images obtained by epifluorescence microscopy after immunodetection of ZO-1 (green) and α-SMA (red). Nuclei were counterstained with DAPI (blue). The other panels show images obtained by differential interference contrast (DIC) (E, F) and bright-field (G) microscopy. In all cases, the HPTCs were cultivated on the bottom of the wells of 24-well plates. (A) First, a well-differentiated epithelial monolayer is formed. (B) Subsequently, myofibroblast aggregates that are strongly positive for α-SMA appear. (C, E) The monolayer then retracts on the one side of the myofibroblast aggregates, leaving a surface devoid of cells (left half in C). (D, F) The monolayer subsequently retracts on the other side of the myofibroblast aggregates. This leads to the formation of cell stripes, which include myofibroblast aggregates (myofibroblast aggregates are labeled with arrowheads in E, F and G). (G, H) Finally, large renal tubules are formed on the 2D solid surface. Several images were stitched together in order to cover the entire tubule shown in G. Scale bars: 100 μm (A), 200 μm (B-F, H), and 1 mm (G).

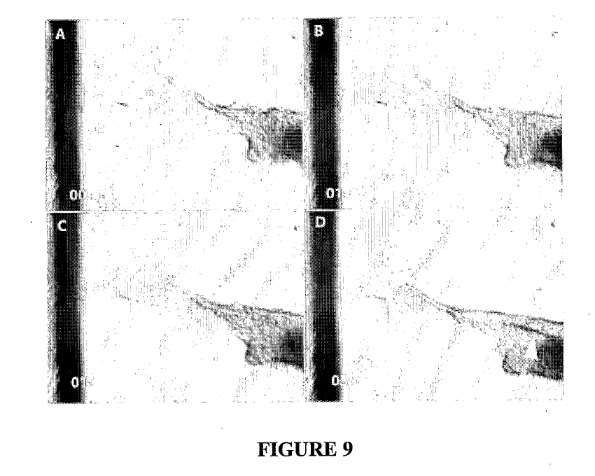

FIG. 9: Tubule formation is associated with rapid cell movements. The panels show the same area imaged by DIC microscopy at consecutive time points (minutes and seconds are indicated in the lower left corner). The images show living HPTCs in cell culture medium on the bottom of a well of a 24-well plate. The imaged area contains part of a myofibroblast aggregate (right edge). The monolayer has already retracted on one side of the myofibroblast aggregate (note upper right area devoid of cells). Cells are in the process of retracting from the other side and folding up the cell stripe into a tubule. The cell stripe is substantially narrowed over the period of 5 min, as indicated for one region marked by the small arrowheads. A tubule-like structure with two clear borders (large arrowheads) is visible at the end of the observation period, but not at the previous time points. Thus, this structure and its lower border (marked by the lower large arrowhead) formed in only about 3 min. Cells at the borders of the stripe (marked by arrow) are quickly integrated into the tubule that is being formed. The dark line on the left side of the panels belongs to a grid, which has been drawn on the outer surface of the well bottom to facilitate spatial orientation during the imaging of cell movements. Scale bar: 200 μm.

FIG. 10: Tubules have a lumen lined by a differentiated epithelium. Cross-section (A) and longitudinal section (B) of a tubule. Tubules were stained with DAPI (white). (C) The surface of a tubule is imaged by epifluorescence microscopy (the upper right areas are out of focus). ZO-1 (white) is detected by immunofluorescence. The tubular epithelium shows extensive formation of tight junctions, as indicated by the chicken wire-like ZO-1 patterns. (D) γGTP activity is detected histochemically. Higher levels of γGTP activity result in the darker staining of cells. The image shows high levels of γGTP activity in a tubule, while the monolayer cells below displays lower levels of activity of this brush border enzyme. Scale bars: 100 μm (A-C) and 200 μm (D).

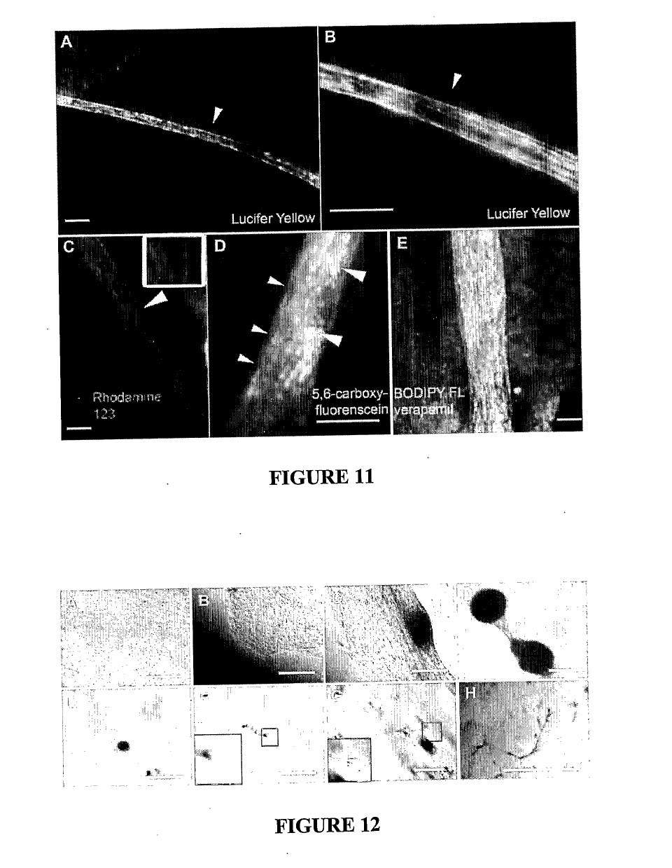

FIG. 11: Organic anion transport. Human proximal tubules formed on 2D solid surfaces were incubated for 20 h with the organic anions lucifer yellow (A, B; green), rhodamine 123 (C; red), 5,6-carboxyfluorescein (D; green) and BODIPY FL verapamil (F; green). Tubules are fixed before imaging, and the cell nuclei are counterstained with DAPI (blue). Panel B shows an enlarged sector of the tubule displayed in A. The arrowhead points to the outer layer of cells lining the tubular lumen, which displays only faint lucifer yellow fluorescence. In contrast, the lumen is strongly labeled. (C) Rhodamine 123 is enriched in the tubular lumen, as compared to the outer layer of cells. The arrowhead points to a region that is enlarged in the inset. The DAPI-stained nuclei of the outer cell layer are on the right in the inset. The cytoplasm displays only very faint rhodamine 123 fluorescence, which is enriched in the tubular lumen (on the left in the inset). (D) The small arrowheads point to the cytoplasm between the DAPI-stained nuclei of the outer cell layer. The cytoplasm displays only faint 5,6-carboxyfluorescein fluorescence. 5,6-carboxyfluorescein is enriched in the tubular interior (large arrowheads). (F) BODIPY FL verapamil is enriched in the cytoplasm of tubular and monolayer cells. Scale bars: 100 μm.

FIG. 12: Tubule formation by HPTCs on 2D solid surfaces and in 3D gels. Panels A-D show tubule formation by HPTCs growing on MATRIGEL-coated bottoms of 24-well plate wells. (A) First, a confluent monolayer is formed. (B). Subsequently, the monolayer retracts on one side. (C) Then the monolayer retracts on both sides of a myofibroblast aggregate. (D) Finally, a tubule attached to myofibroblast aggregates is formed. The process is similar to that shown in FIG. 8. Panels E-H show tubule formation by HPTCs suspended in MATRIGEL. (E) Initially, single cells or small groups of cells are present. Note that most of these structures distributed in the 3D gel are out of focus, if a given field is imaged and appear as blurred rings on the images. (F, G) Cell outgrowth occurs (no cyst formation before cell outgrowth), leading to the formation of elongated cords or tubules. The tip cells are typically branched and display multiple filopodia (shown as enlarged in the insets; the branched cell shown in F appears blurred due to problems with imaging these structures within the gel). (H) Finally, thin tubules displaying multiple branches are formed. The size of tubules formed in matrigel is typically less than 1 mm, and the tubules are not attached to myofibroblast aggregates (note the different morphology of the structures shown in panels D and H). Scale bars: 1 mm (A-D), 100 μm (F-G) and 500 μm (H).

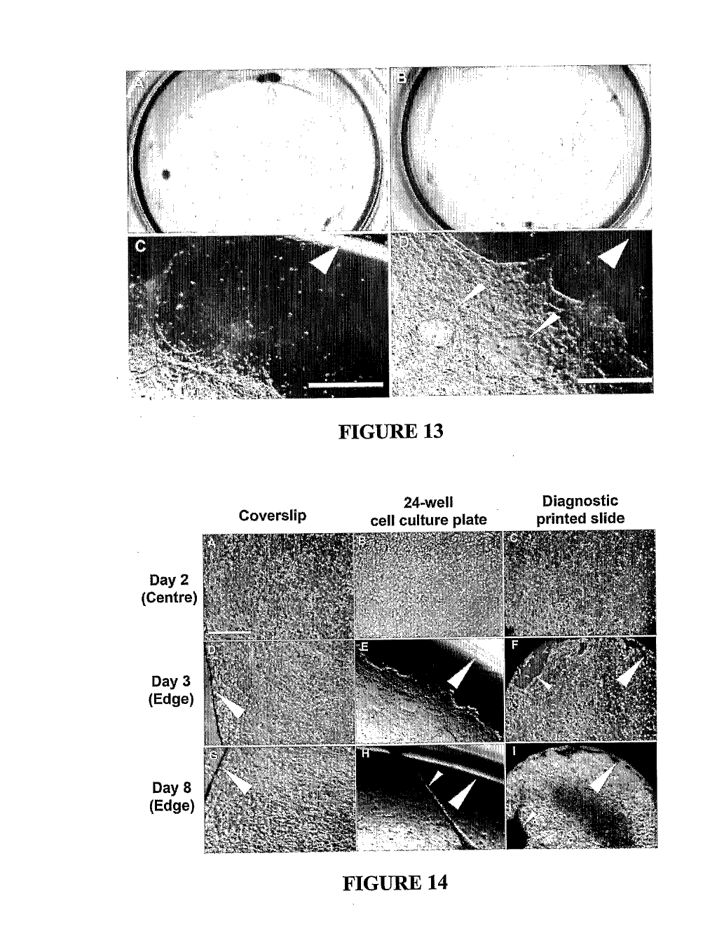

FIG. 13: Sensing of a 3D edge triggers tubulogenesis. A and B show two wells of 24-well plates with HPTCs. Multiple tubules with attached myofibroblast aggregates (two of these structures are marked by arrows) are present within these wells (well diameter=15 mm). The tubules always display a similar distance from the edge, which leads to the generation of ring-like structures consisting of tubules. C and D show initial retraction of the monolayer starting at the edge the wells. Uneven illumination is due to optical effects at the edge. The direction where the edge is located is indicated by large arrowheads, and part of the edge is visible in the upper right corner in C. A part of the monolayer is visible in the lower left corner in C. All cells of the monolayer moved simultaneously from the edge towards the center, leaving an almost void surface behind. Panel D shows a cell layer that retracted from the edge. Here, coordinated retraction from the opposite side has started, which breaks up the cell layer (marked by small arrowheads) at defined distances from the outer rim. Scale bars in C and D: 500 μm.

FIG. 14: Triggering of tubulogenesis depends on the presence of a 3D substrate architecture. HPTCs were grown to confluency on glass coverslips (A, D and G), in the wells of 24-well plates consisting of tissue culture plastic (B, F and H) and in the wells of diagnostic printed slides (C, F and I). Coverslips with a side length of 18 mm are used. The wells of 24-well plates and diagnostic printed slides are 15 mm and 2 mm in diameter, respectively. Cells on the different devices are monitored over a time period of 8 days. Panels A-C show the confluent monolayers at day 2 in the centers of the coverslips or wells. (E, F) Monolayer retraction starts at day 3 at the edges of the wells (marked by large arrowheads) of 24-well plates and diagnostic printed slides. This leads to areas devoid of cells (marked by a small arrowhead in F). No rearrangements are observed at (D) day 3 and (G) day 8 at the edges of coverslips (marked by large arrowheads), which do not have a 3D structure. The monolayer is still intact on coverslips. In contrast, major rearrangements have taken place at day 8 in the wells of (H) 24-well plates and (I) diagnostic printed slides. Formation of tubule (marked by small arrowhead in H) and myofibroblast aggregates (marked by small arrowhead in I) has occurred. Note that the wells of 24-well plates and diagnostic printed slides provide different surface chemistries and surface areas. However, in both cases, the edge (marked by large arrowheads in E, F, H and I) is a 3D structure, in contrast to the edge of coverslips. Scale bar: 500 μm.

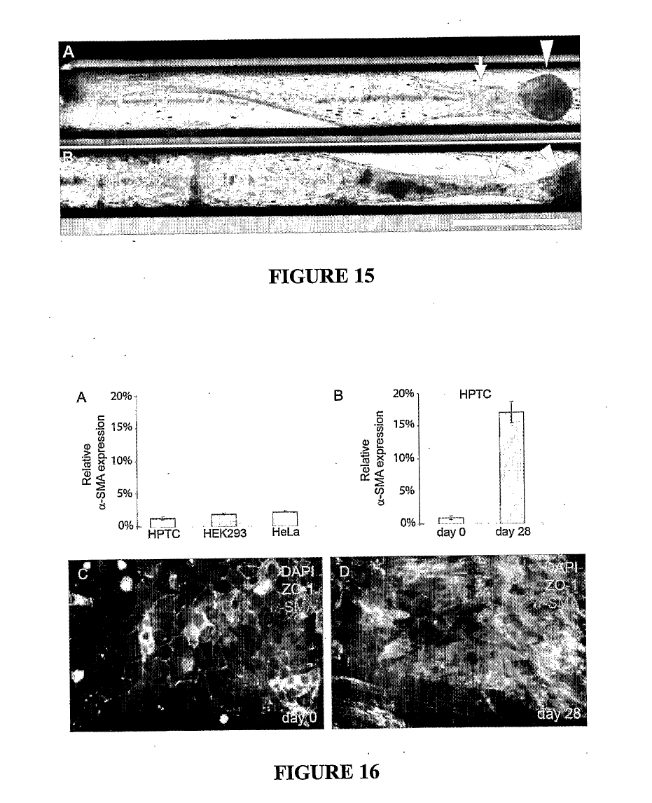

FIG. 15: Tubulogenesis in capillaries. HPTCs are seeded into glass capillaries with an inner diameter of 580 μm. A and B show 2 different capillaries containing HPTCs imaged 2 weeks after seeding. Several images were stitched together in order to cover a larger area. Initially after seeding, monolayers covering the inner walls of the capillaries are formed. The monolayer is still intact in the left half of the lower capillary (B). Myofibroblast aggregates appear after monolayer formation. The monolayer is then rearranged and detached from the capillary walls, and tubules are formed within the capillaries (marked by arrows), which are attached to myofibroblast aggregates (marked by arrowheads). Scale bar: 1 mm.

FIG. 16: α-SMA expression in initial and 4 week-old cultures of HPTCs. (A) The expression levels of α-SMA (relative to GAPDH, average+/−s.d.) were determined by qRT-PCR in initial cultures of HPTCs. These initial cultures contained cells freshly seeded from the vial obtained by the vendor and the cells had not been passaged before analysis. The analysis was performed as soon as an epithelial sheet had been formed. For comparison, similar qRT-PCR analyses were also performed with confluent monolayer cultures of HEK293 and HeLa cells and the results are shown. (B) α-SMA expression (relative to GAPDH, average+/−s.d.) was determined by qRT-PCR in initial cultures of HPTCs (day 0) and 28 days later in cultures which were seeded in parallel. The cultures were not passaged during this time period but the medium was regularly exchanged. (C) The image shows an initial culture of HPTCs (day 0) after co-immunostaining (ZO-1: green, α-SMA: red, DAPI: blue). α-SMA was not detectable. (D) The same co-immunostaining procedure was performed after 28 days with cultures seeded in parallel. Many α-SMA-expressing cells are present.

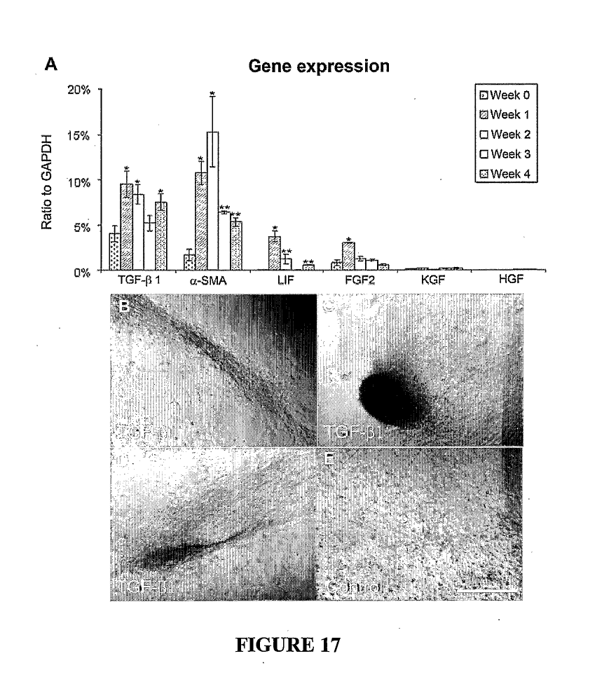

FIG. 17: Growth factor expression and effects of TGF-β1. (A) The expression levels of TGF-β1, α-SMA, LIF, FGF2, KGF and HGF are monitored over a period of 4 weeks. The expression levels are determined by quantitative RT-PCR, and displayed as percentages of GAPDH expression. The five different bars displayed for each factor show the relative expression levels (average+/−standard deviation) at day 1 (week 0) and at weeks 1-4 after seeding. Results that are significantly different (p<0.05) from the data obtained at day 1 are marked with an asterisk. Results that are significantly different (p<0.05) from the data obtained at day 1 and at week 1 are marked with two asterisks. Panels B-D show the cells treated for 3 days with 10 ng/ml TGF-β1 after monolayer formation. TGF-β 1 treatment induces rearrangements leading to the formation of condensed stripes of cells and areas devoid of cells (B, D). Panel C shows a cell aggregate. Panel F displays the untreated control cells, whereby the intact monolayer is maintained. Scale bar: 500

### Results

Human Renal Tubule Formation on 2D Solid Surfaces

Initial formation of a flat and well differentiated epithelial monolayer was observed when HPTCs were cultivated in multi-well plates (FIG. 8 A). Subsequently, increasing amounts of α-SMA-expressing myofibroblasts appeared. These myofibroblasts formed large aggregates (FIG. 8 B). In the surroundings of such aggregates, the epithelium became reorganized. Firstly, highly coordinated and simultaneous directed movements of large numbers of cells led to retraction of the monolayer on one side of myofibroblast aggregates, leaving behind the largely empty surface of the well (FIG. 8 C, E). Subsequently, the monolayer retracted on the other side of the myofibroblast aggregates (FIG. 8 D, F). These highly coordinated cell movements led to the formation of stripes of cells, with a length of up to several millimeters or even centimeters (FIG. 8 F). The stripes included the myofibroblast aggregates.

The cells that have organized into a stripe then performed additional dynamic reorganizations, which gave rise to tubule formation (FIG. 8 G, H). The human renal tubules formed in this way on 2D solid surfaces were straight and not branched, and typically have a length of several millimeters (FIG. 8 G). The tubules always remained attached to myofibroblast aggregates, which could be associated with one (FIG. 8 G) or both ends of a tubule but could also be found at mid-tubular regions. When an end of a tubule was not attached to a myofibroblast aggregate the tubular epithelium was continuous with the remainder of the monolayer (FIG. 8 G, H). The finally formed tubular epithelium and attached epithelia were well differentiated (FIGS. 8 H and 10 C, D).

Cell movements involved in monolayer reorganization and tubule formation were not only highly coordinated, but also rapid. FIG. 9 shows that condensation and folding up of the cell stripe, which ultimately led to tubule formation, occurred within minutes.

Tubules have a Lumen Lined by a Differentiated Epithelium and Display Transport Functions

Sectioning of the tubules formed by the processes described above confirmed that the tubules enclosed a lumen (FIG. 10 A, B). The lumen was lined by a well-differentiated epithelium displaying extensive tight junction formation (FIG. 10 C). The brush border marker γ-glutamyl transpeptidase (γGTP) was expressed within the tubules (FIG. 10 D), confirming cell type-specific differentiation and apical-basal polarity of the epithelial cells.

In order to test whether the tubular epithelium displayed typical transport functions, tubules were incubated with the fluorescent organic anions lucifer yellow, rhodamine 123,5,6-carboxyfluorescein and BODIPY FL verapamil. Lucifer yellow and 5,6-carboxyfluorescein are substrates of the p-aminohippurate transport system. Stronger fluorescence was observed within the tubular lumen, as compared to the surrounding medium and the epithelial cells lining the lumen. Thus, these organic anions became enriched in the tubular lumen (FIG. 11), providing evidence for transport across the tubular epithelium. Also rhodamine 123, which is a substrate of the multidrug resistance-1-encoded P-glycoprotein (P-gp) transport system, became enriched in the tubular lumen (FIG. 11). Rhodamine 123 is actively transported, whereas BOPIPY FL verapamil is transported by the P-gp system via electrodiffusive anion transport. BOPIPY FL verapamil became enriched within the cells (FIG. 11), suggesting transport of this substrate at the basolateral sites into the cells, but slower or no transport at their apical sites.

Whether this reflected impairment of the P-gp mediated electrodiffusive anion transport at the apical sites of the cells was not clear, as the kinetics and exact routes of BODIPY FL verapamil transport in the native human proximal tubule were not characterized.

Together, the results showed that various organic anions were transported across the tubular epithelium, and suggested that at least two different major transport pathways were functional.

Tubule Formation by HPTCs on 2D Solid Surfaces and in 3D Gels

In order to address tubule formation by HPTCs in 3D gels the cells were cultivated in MATRIGEL™. Tubule formation in MATRIGEL™ (FIG. 12 E-H) involved branching of cells at the initial stages of tubule formation and outgrowth of branched cells. Outgrowing branches then formed tubules, and budding from these tubules could occur, giving rise to branched tubular structures (FIG. 12 H). It was important to note that formation of epithelial monolayers and coordinated movements of large numbers of cells were not involved in tubule formation in 3D gels. The resulting human proximal tubules obtained in 3D gels were relatively small, displayed multiple branches (FIG. 12 H), could be convoluted (FIG. 7), and were never attached to myofibroblast aggregates, in contrast to the tubules obtained on 2D solid surfaces.

In order to test whether MATRIGEL™ had an influence on the process of tubulogenesis, tubule formation was investigated with MATRIGEL™-coated multi-well plates, where HPTCs grew on top of the MATRIGEL™ coating. Under these conditions, tubule formation occurred in a similar way as observed before on 2D solid surfaces (FIG. 12 A-D). Generally, different extracellular matrix (ECM) coatings consisting of laminin, collagen IV, a mixture of these components or other components could influence the timing of monolayer reorganization, and the extent of myofibroblast aggregate formation.

When tubules were formed on 2D solid surfaces, they always occurred by the same process as illustrated in FIGS. 8 and 12 A-D, regardless of the ECM coating used. Tubulogenesis occurred also on uncoated 2D solid surfaces. Together, the results showed that HPTCs form small tubules in 3D gels by a process of budding and branching morphogenesis, whereas large tubules are formed on 2D solid surfaces by a process involving large-scale reorganizations of epithelial monolayers and interactions between epithelial cells and myofibroblasts.

Tubule Formation on 2D Solid Surfaces is Enhanced by a Curved Surface Architecture

Although renal tubule formation occurred on 2D solid surfaces, several observations suggested that formation of 3D tissue-like structures by HPTCs was enhanced by an orthogonal substrate architecture. Most striking was the finding that a closed circle formed by several tubules with attached myofibroblast aggregates could be formed within a well close to its edge, with all tubules displaying a similar distance to the edge (FIG. 13 A, B). In contrast to the center of the well, the edge of the well has an orthogonal architecture (i.e. a perpendicular wall). Another observation suggesting an important role for an orthogonal architecture in enhancing tubule formation was that initial retraction of the epithelial monolayer started in most cases first at the edge of the well (FIG. 13 C, D).

In order to determine whether the presence of a perpendicular edge indeed enhanced the initiation of tubule formation, cells were seeded in parallel on 18 mm coverslips (no perpendicular edge), 24-well cell culture plates (with perpendicular edge), and diagnostic printed slides (with perpendicular edge). FIG. 14 shows that initial retraction of the epithelium first occurred at the edges of the wells of the 24-well plates (well diameter 15 mm) and diagnostic printed slides (well diameter 2 mm). Subsequently, monolayer reorganization as well as cell aggregate and tubule formation was observed within the wells of these devices. In contrast, the epithelial monolayer was not reorganized on coverslips during the monitoring period of 8 days, and no retraction of the monolayer occurred at the edges of the coverslips (FIG. 14).

The surface material was glass in the case of coverslips and diagnostic printed slides, whereas 24-well plates consisted of tissue culture plastic. Furthermore, their sizes were different, and thus the numbers of cells that could be involved in reorganization processes on the different surfaces were also different. The results showed that tubule formation was not dependent on the material, surface area and cell numbers involved, but was only dependent on the presence of a perpendicular edge.

In order to find out whether cells might sense the rigid wall, HPTCs were seeded into glass capillaries. In glass capillaries, HPTCs could only attach with their basal sides to the rigid substrate, but not with their lateral sides. Furthermore, it was important to find out whether the process of tubule formation might be inhibited if the cells were already arranged into a tubular architecture.

FIG. 15 shows that HPTCs formed tubules within capillaries. These results demonstrated that tubule formation was not inhibited by arranging HPTCs into a pre-formed tubular architecture. Tubule formation within capillaries was accomplished by the same process as employed on 2D solid surfaces, involving monolayer formation and subsequent appearance of myofibroblast aggregates. The results also revealed that lateral attachment to a rigid substrate was not important for the sensing of a 3D environment, which was provided by the edges of the wells as well as by glass capillaries.

TGF-β1 Induces the Initial Steps of Human Renal Tubule Formation on 2D Solid Surfaces

Besides substrate architecture, interactions between epithelial cells and myofibroblasts appeared to be important for tubule formation. FIG. 16 C shows that α-SMA-expressing cells were not detectable by immunostaining in the initial cultures of HPTCs. The expression levels of α-SMA as determined by quantitative real-time polymerase chain reaction (qRT-PCR) were very low in such initial cultures and were not higher than the expression levels in HeLa cells or human embryonic kidney (HEK) 293 cells (FIG. 16 A). HeLa cells are negative for α-SMA and HeLa as well HEK293 cells are well established epithelial cell lines free of contaminations with other cell types. In contrast, large amounts of α-SMA-expressing cells could be detected by immunostaining after maintaining such initially myofibroblast-free HPTC cultures for four weeks under in vitro conditions (FIG. 16 D). Accordingly, the expression levels, as detected by quantitative RT-PCR, increased (FIG. 16 B). As kidney epithelial cells can transdifferentiate into myofibroblasts under in vitro conditions by an epithelial-to-mesenchymal-transition (EMT) process, the most likely explanation for the appearance of myofibroblasts in the initially myofibroblast-free HPTC cultures is transdifferentiation of epithelial cells into myofibroblasts by an EMT process. At least the data show that the initial cultures of HPTCs were not contaminated with α-SMA-expressing myofibroblasts.

As tubulogenesis was initiated in the vicinity of myofibroblast aggregates, the types of myofibroblast-derived signaling molecules expressed in the in vitro cultures were examined (FIG. 17 A). The expression levels of myofibroblast-derived growth factors were determined by qRT-PCR in parallel with the expression levels of the myofibroblast marker α-SMA. In vitro cultures were monitored over a time period of 4 weeks. Typically, myofibroblast aggregates appeared after 1-2 weeks of in vitro culture. Cell loss could occur in the course of reorganization events due to detachment of huge cell aggregates and epithelial sheets.

The results revealed that keratinocyte growth factor (KGF) and hepatocyte growth factor (HGF) were not expressed or expressed at very low levels. In contrast, substantial expression of TGF-β1, leukemia inhibitory factor (LIF) and fibroblast growth factor (FGF)₂ was observed, along with the expression of α-SMA. In all of these cases, a massive increase in expression levels was observed after 1 week of in vitro culture. This was in accordance with the observation that myofibroblasts appeared after 1-2 weeks of in vitro culture, and the expression levels of α-SMA and TGF-β1 remained high until the second week. The expression levels of LIF and FGF2 dropped after week 1, suggesting down regulation. The levels of TGF-β1 and α-SMA expression were significantly lower at week 3 (as compared to week 2), and this might reflect down regulation and/or cell loss. The expression levels of TGF-β1 significantly increased again at the end of the monitoring period (week 4).

It has been described before that TGF-β1 triggers the initial steps of tubule formation occurring on 2D solid surfaces, namely the formation of a condensed stripe or cord of cells from a monolayer. As these previous experiments were performed with rabbit cells, we tested here whether primary human cells reacted in a similar way. Indeed, treatment with TGF-β1 led to rearrangement of the monolayer into a condensed stripe of cells and to the formation of cell aggregates (FIGS. 17 B-E). This result showed that TFG-β1 induced the initial steps of human renal tubule formation on 2D solid surfaces.

### Example 4

Experiments were performed to compare the method of Humes et al. (Humes and Cieslinski Exp. Cell Res. 1992 201: 3-15; U.S. Pat. No. 5,429,938) with the present method for the formation human proximal tubules.

The Humes et al. method used primary rabbit proximal tubule cells. The references indicate three factors are necessary in the growth medium for 2D tubulogenesis: (i) transforming growth factor (TGF)-β1; (ii) epidermal growth factor (EGF); and all-trans retinoic acid (RA). The method involves cultivation of primary rabbit renal proximal tubule cells in the following medium: Dulbecco's modified Eagle's:Ham's F-12 media (1:1, v/v) containing L-glutamine, penicillin, streptomycin, 50 nM hydrocortisone, 5 μg/ml of insulin, and 5 μg/ml of transferrin, referred to herein as Humes basal medium.

As described in the references, the rabbit proximal tubule cells became confluent after 9-12 days in the Humes' basal medium. Addition of TGF-β1 (10 ng/ml), EGF (1 nM) and RA (0.1 μM=100 nM) to such confluent cultures for at least 72 hours led to the formation of kidney tubules on 2D solid surfaces. Addition of all three factors was required for tubulogenesis. Addition of only TGF-β1 led to the formation of solid aggregates of adherent cells (no lumen). When TGF-β1 and EGF were added these solid aggregates were larger (also no lumen formation). Simultaneous exposure to TGF-β1 and RA led to the formation of aggregates with primordial lumens, which only fully developed in the presence of EGF, likely due to the expansion of lining epithelial cells. Thus, the different factors appear to have the following effects on rabbit cells: TGF-β1 (formation of solid aggregates); EGF (mitotic cell growth); RA (cell differentiation (epithelial) and initiation of lumen formation).

It appears, based on the Humes et al. results, that the formation of solid aggregates induced by TGF-β1 is an initial event in tubulogenesis, as RA and EGF alone had no dramatic effects on the monolayer. EGF does not appear to play any specific role in tubulogenesis, but mainly affects cell proliferation, which has an indirect effect on tubule morphology. It is well-known that EGF acts as a mitogen on rabbit proximal tubule cells.

Using the methods of the present invention and HPTCs, tubule formation by HPTCs on solid surfaces has been observed in the following culture medium: basal epithelial cell medium (ScienCell Research Laboratories, Carlsbad, Calif., USA; containing salts, sugars, amino acids etc.) supplemented with 2% fetal bovine serum (FBS), apo-transferrin (10 μg/ml), insulin (5 μg/ml), hydrocortisone (1 μg/ml), epinephrine (500 ng/ml), fibroblast growth factor (FGF) (2 ng/ml), EGF (10 ng/ml) and RA (10 nM). The medium also contained penicillin and streptomycin.

It should be noted that no TGF-β1 was added as a supplement or was included in the growth medium as a component. It is possible that some trace amount of contaminating TGF-β1 was present in the FBS. As the highest concentrations of this growth factor measured in FBS are reported to be in the range of 16 ng/ml, it would be expected that the TGF-β1 concentration in the medium used in the HPTC experiments was maximally 0.3 ng/ml or less. This is much lower than the concentration of TGF-β1 applied to the rabbit cells (10 ng/ml) in the Humes et al. method.

The formation of condensed cell aggregates also played an important role in 2D tubule formation with the HPTCs. After the formation of a confluent monolayer of epithelial cells, the monolayer became reorganized into 2D tubules. Condensed cell aggregates were central in these reorganization processes. Obviously, the formation of these cell aggregates was not dependent on the addition of purified TGF-β1, as suggested by the results obtained in the Humes et al. method with rabbit cells. During the process of tubule formation according to the present method using HPTCs, the cell aggregates consisted of αSMA-expressing myofibroblasts, which likely arose by trans-differentiation of the epithelial cells into this cell type (epithelial-to-mesenchymal transition). It is well known that TGF-β1 plays a central role in this process and promotes trans-differentiation of epithelial cells into myofibroblasts (mesenchymal cell type). Thus, it is possible that trans-differentiation-dependent formation of cell aggregates, which appears to be crucial for 2D tubule formation, was enhanced under the conditions used by Humes et al. by the addition of high concentrations of TGF-β1. However, contrary to the disclosure by Humes et al., addition of TGF-β1 may not be required, given that kidney cells are able to secrete TGF-β1 themselves, thus directing the trans-differentiation processes and tissue reorganization.

The following experiments were conducted to determine if addition of TGF-β1, EGF and RA are really a requirement. Also addressed was whether the conditions described by Humes et al., which lead to tubule formation by rabbit kidney cells, can be also applied to HPTCs and result in functional tubules. All of the experiments described below were performed with HPTCs.

Experimental Results

In a first set of experiments the cells were cultivated in Humes basal medium (see above), not supplement with TGF-β1, EGF, or RA. 4 series of experiments were performed with two different batches of HPTCs (derived from 2 different patients). In all experimental series, extensive initial cell death was observed. This shows that the medium suitable for cultivation and propagation of the rabbit kidney cells is suboptimal for HPTCs. Two of the experimental series were terminated prematurely due to the observed massive cell death. Two of the series were continued and in one of these series all cells were dead after 12 days. However, some cells survived and divided in the other series and this series comprised 10 wells seeded with cells. In these wells some confluent patches of cells developed and 2D tubules appeared in 6 out of 10 wells.

These results demonstrated that the medium applied to the rabbit cells is suboptimal for HPTCs and in most of the cases HPTCs die. As well, addition of TGF-β1, EGF and RA is not essential for 2D tubule formation by HPTCs. Thus, the conditions used for rabbit proximal tubule cells are not conducive for the formation of 2D tubules by HPTCs.

In a second set of experiments cells were again cultivated in Humes basal medium. In accordance with the previous results, massive cell death occurred although some cells survived and formed some confluent patches after 2 weeks. By this time cell growth was very slow and overall confluency could not be achieved. However, the confluent patches were treated with 10 ng/ml TGF-β1 in accordance with the Humes et al. method. The TGF-β1 had no obvious effect on the monolayer and no cord and tubule formation was observed. One explanation for these results might be that a functionally different cell population was enriched amongst the surviving cells. At least these results show again that the conditions used for rabbit proximal tubule cells are different than those described herein for the formation of tubules on 2D solid surfaces.

In a third set of experiments comprising 6 experimental series, the HPTC epithelial medium with all supplements except FBS was used. Also here cell death was observed to a variable degree. 2D tubule formation was never observed under these conditions.

These results suggest that addition of FBS is required for optimal propagation and for 2D tubule formation by HPTCs.

A fourth experimental series was performed with our HPTC medium containing all supplements including FBS. HPTCs were grown to confluency and then treated with TGF-β1 (10 ng/ml; same concentration as used for the rabbit cells). After three days massive cords and aggregates of cells with an elongated, myofibroblastic morphology had formed in 6/6 wells. Although in the complete HPTC medium with FBS, cords and cell aggregates also form without the addition of extra TGF-β1, such structures formed more consistently after addition of TGF-β1. Of note, the morphology of the cords was different compared to the cord-/tubule-like structures observed without TGF-β1 and they appeared to represent more massive aggregates of myofibroblastic cells.

This result is in agreement with the results obtained by Humes et al. with rabbit cells and suggests that TGF-β1 promotes the formation of massive cell aggregates, consisting mainly of non-polarized mesenchymal cells. Thus, addition of exogenous TGF-β1 appears unnecessary and in fact results in improperly formed tubules generated by HPTCs.

Of note, Humes et al. report that the majority of cells in the tubular structures formed by rabbit kidney cells displayed mesenchymal character. The present results suggest that this effect is due to the presence of high concentrations of TGF-β1. As outlined above, this gives rise to tubular structures submerged within masses of mesenchymal cells, which are less optimal for various applications than the mainly epithelial tubules obtained using the methods described herein. Thus, it appears that addition of high concentrations of purified TGF-β1 is not only not required, but even gives rise to the formation of suboptimal tubular structures.

Discussion

Tubules formed by the Humes et al. method are formed from thick aggregates of mainly non-polarized, adherent mesenchymal cells. Slit-like lumens form within these cell masses, displaying a width of less than one cell diameter. Only the few cells bordering these small lumens display epithelial differentiation and polarization. Thus, the tubule with its walls of epithelial cells is submerged within masses of mesenchymal cells.

In contrast, the 2D tubules generated by the methods described herein display extensive lumen formation and the walls of the 2D tubules consist of differentiated epithelia expressing tight junctions and brush border markers. Some mesenchymal cells (myofibroblasts) are typically attached to these epithelia but they do not form condensed cell masses surrounding the tubules. Thus, the lining epithelia are not submerged within other cell masses and are directly exposed to the environment.

In vivo, the walls of proximal tubules consist of an epithelium, which lines a coherent lumen with a diameter of at least about 65 μm (about 1 cell diameter). The proximal tubules are surrounded by only some interstitial fibroblasts. Thus, under normal conditions proximal tubules are not embedded into condensed masses of non-polarized adherent cells. Together, the findings suggest that the morphology of the 2D tubules generated by the presently described methods is more similar to the morphology of native proximal tubules.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

All technical and scientific terms used herein have the same meaning as commonly understood by one of ordinary skill in the art of this invention, unless defined otherwise.

Any sub-range, sub-list or sub-combination included within any range, list or combination set out herein is intended to be included as if the sub-range, sub-list or sub-combination was expressly specified.

As used in this specification and the appended claims, the singular forms “a”, “an” and “the” include plural reference unless the context clearly dictates otherwise. As used in this specification and the appended claims, the terms “comprise”, “comprising”, “comprises” and other forms of these terms are intended in the non-limiting inclusive sense, that is, to include particular recited elements or components without excluding any other element or component. Unless defined otherwise all technical and scientific terms used herein have the same meaning as commonly understood to one of ordinary skill in the art to which this invention belongs.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

### REFERENCES

U.S. Pat. No. 5,429,938
Ryan M J, Johnson O, Kirk I, Fuerstenberg S M, Zager R A, Torok-Storb B. HK-2: an immortalized proximal tubule epithelial cell line from normal adult human kidney. Kidney Int 1994; 45(1):48-57.
Beacham D A, Amatangelo M D, Cukierman E. Preparation of extracellular matrices produced by cultured and primary fibroblasts. Curr Protoc Cell Biol 2006; Supplement 33(Chapter 10:Unit 10.9): 10.9.1-10.9.21.
Sadoni N, Langer S, Fauth C, Bennardi O, Cremer T, Turner B M, et al. Nuclear organization of mammalian genomes: polar chromosome territories build up functionally distinct higher order compartments. J Cell Biol 1999; 146(6): 1211-1226.
Karihaloo A, Nickel C, Cantley L G. Signals which build a tubule. Nephron Exp Nephrol 2005; 100(1):e40-45.
Schumacher K M, Phua S C, Schumacher A, Ying J Y. Controlled formation of biological tubule systems in extracellular matrix gels in vitro. Kidney Int 2008; 73(10): 1187-1192.
Han H J, Sigurdson W I, Nickerson P A, Taub M. Both mitogen activated protein kinase and the mammalian target of rapamycin modulate the development of functional renal proximal tubules in matrigel. J Cell Sci 2004; 117(Pt 9):1821-1833.
Takemura T, Hino 5, Okada M, Murata Y, Yanagida H, Ikeda lvi, et al. Role of membrane-bound heparin-binding epidermal growth factor-Like growth factor (HB-EGF) in renal epithelial cell branching. Kidney Int 2002; 61(6): 1968-1979.
Humes H D, Cieslinski D A. Interaction between growth factors and retinoic acid in the induction of kidney tubulogenesis in tissue culture. Exp Cell Res 1992; 201(1):8-15.
Lubarsky B, Krasnow M A. Tube morphogenesis: making and shaping biological tubes. Cell 2003; 112: 19-28.
Montesano R, Matsumoto K, Nakamura T, et al. Identification of a fibroblast derived epithelial morphogen as hepatocyte growth factor. Cell 1991; 67: 901-908.
Montesano R, Schaller G, Orci L. Induction of epithelial tubular morphogenesis in vitro by fibroblast-derived soluble factors. Cell 1991; 66: 697-711.
Nickel C, Benzing T, Sellin L, et al. The polycystin-1 C-terminal fragment triggers branching morphogenesis and migration of tubular kidney epithelial cells. J Clin Invest 2002; 109: 481-489.
Sakurai H, Barros E J, Tsukamoto T, et al. An in vitro tubulogenesis system using cell lines derived from the embryonic kidney shows dependence on multiple soluble growth factors. Proc Natl Acad Sci USA 1997; 94: 6279-6284.
Taub M, Wang Y, Szczesny T M, et al. Epidermal growth factor or transforming growth factor alpha is required for kidney tubulogenesis in matrigel cultures in serum-free medium. Proc Natl Acad Sci USA 1990; 87: 4002-4006.
Zegers M M, O'Brien L E, Yu W, et al. Epithelial polarity and tubulogenesis in vitro. Trends Cell Biol 2003; 13: 169-176.
Zhang H, Tasnim F, Ying J Y, et al. The impact of extracellular matrix coatings on the performance of human renal cells applied in bioartificial kidneys. Biomaterials 2009; 30: 2899-2911.

1. A method of making a renal tubule, the method comprising:
seeding renal tubule cells onto a solid surface;
culturing the renal tubule cells in a liquid growth medium to form a monolayer on the solid surface; and
continuing culturing the renal tubule cells to form a tubule, the resulting tubule having an unbranched or minimally branched morphology, a length of from about 0.1 mm and an interior lumen surrounded mostly by differentiated epithelial cells.

2. The method of claim 1 wherein the renal tubule cells are primary renal tubule cells, cells from a cultured cell line, embryonic primary kidney cells, kidney precursor cells, cells differentiated from embryonic stem cells, cells differentiated from mesenchymal stem cells or cells differentiated from induced pluripotent stem cells.

3. The method of claim 1 wherein the renal tubule cells are proximal tubule cells, distal tubule cells or collecting duct cells.

4. The method of claim 2 wherein the primary renal tubule cells are human primary renal proximal tubule cells.

5. The method of claim 1 wherein the solid surface is concave.

6. The method of claim 1 wherein the solid surface has an intersecting wall.

7. The method of claim 6 wherein the solid surface is a patterned surface having multiple intersecting walls.

8. The method of claim 1 wherein the liquid growth medium comprises 0.5% (v/v) or greater serum.

9. The method of claim 1 wherein the solid surface is coated with an extracellular matrix or an extracellular matrix component.

10. The method of claim 1 further comprising addition of a test compound prior to tubule formation.

11. An in vitro generated renal tubule formed on a solid surface and having an unbranched or minimally branched morphology, a length of from about 0.1 mm and an interior lumen surrounded mostly by differentiated epithelial cells.

12. The in vitro generated renal tubule of claim 11 further having one or more of the following properties:
(i) a length of from about 0.1 mm to about 1.5 cm;
(ii) the interior lumen having a diameter of from about 1 μm to about 200 μm; and
(iii) renal uptake and transport functions.

13. The in vitro generated renal tubule of claim 11 that is a human in vitro renal tubule.

14. The in vitro generated renal tubule of claim 11 when prepared according to the method of claim 1.

15. A method of monitoring tubular function, the method comprising:
contacting a compound or particle with the exterior surface of an in vitro renal tubule prepared according to the method of claim 1; and
(i) detecting the compound or particle within the tubule; or (ii) assessing the effect of the compound or particle on the tubule or tubule cells; or both.

16. The method of claim 15 wherein the compound or particle is labeled with a detectable label.

17. The method of claim 15 further comprising incubating the compound or particle with the tubule prior to said detecting.

18. The method of claim 15 further comprising contacting an inhibitor with the exterior of the in vitro renal tubule.

19. The method of claim 18 wherein the compound or particle is a control compound or control particle, and the inhibitor is a test inhibitor.

20. The method of claim 15 wherein said assessing the effect of the compound or particle on the tubule or tubule cells comprises assessing toxicity of said compound or said particle on said tubule or tubule cells.


SEQUENCE LISTING (14 sequences)

SEQ ID NO: 1 - DNA, 21 bp, artificial
  synthetic primer
atcaccatcttccaggagcga

SEQ ID NO: 2 - DNA, 21 bp, artificial
  synthetic primer
ccaaagttgtcatggatgacc

SEQ ID NO: 3 - DNA, 20 bp, artificial
  synthetic primer
tcatcaccaactgggacgac

SEQ ID NO: 4 - DNA, 20 bp, artificial
  synthetic primer
atgctcttcaggggcaacac

SEQ ID NO: 5 - DNA, 20 bp, artificial
  synthetic primer
acctgaacccgtgttgctct

SEQ ID NO: 6 - DNA, 20 bp, artificial
  synthetic primer
gctgaggtatcgccaggaat

SEQ ID NO: 7 - DNA, 20 bp, artificial
  synthetic primer
tacgccacccatgtcacaac

SEQ ID NO: 8 - DNA, 20 bp, artificial
  synthetic primer
tgtccaggttgttggggaac

SEQ ID NO: 9 - DNA, 20 bp, artificial
  synthetic primer
tgtgctaaccgttacctggc

SEQ ID NO: 10 - DNA, 20 bp, artificial
  synthetic primer
gcccaggtcctgttttggat

SEQ ID NO: 11 - DNA, 20 bp, artificial
  synthetic primer
agtggcagttcggattgtgg

SEQ ID NO: 12 - DNA, 20 bp, artificial
  synthetic primer
cccctccattgtgtgtccat

SEQ ID NO: 13 - DNA, 20 bp, artificial
  synthetic primer
tgtccacggaagaggagatg

SEQ ID NO: 14 - DNA, 20 bp, artificial
  synthetic primer
agccccagccataaacactg